(19) United States
(12) Patent Application Publication
(10) Pub. No.: US 2025/0172540 A1
(43) Pub. Date: May 29, 2025

(54) Methods And Systems For Quantifying Partitioning Of Agents In Vivo Based On Partitioning Of Agents In Vitro

(71) Applicant: Whitehead Institute for Biomedical Research, Cambridge, MA (US)

(72) Inventors: Henry R. Kilgore, Boston, MA (US); Kalon J. Overholt, Somerville, MA (US); Richard A. Young, Weston, MA (US)

(21) Appl. No.: 18/859,605

(22) PCT Filed: Apr. 21, 2023
(86) PCT No.: PCT/US2023/066078

§ 371 Date: Oct. 24, 2024

Related U.S. Application Data
(60) Provisional application No. 63/363,572, filed on Apr. 25, 2022.
(60) Provisional application No. 63/476,084, filed on Dec. 19, 2022.

(51) Int. Cl. G01N 33/50; G16B 15/30; G16B 40/20

(52) U.S. Cl. CPC G01N 33/5011; G16B 15/30; G16B 40/20

(57) ABSTRACT

Small molecule therapeutics can concentrate in distinct intracellular environments, some bounded by membranes, and others that may be formed by membrane-less biomolecular condensates. The chemical environments within biomolecular condensates have been proposed to differ from those outside these bodies, but the internal chemical environments of diverse condensates have yet to be explored. Here we use small molecule probes to demonstrate that condensates formed in vitro with the scaffold proteins of different biomolecular condensates harbor distinct chemical solvating properties. The chemical rules that govern selective partitioning in condensates, which we term condensate chemical grammar, can be ascertained by deep learning, allowing efficient prediction of the partitioning behavior of small molecules. The rules learned from in vitro condensates were adequate to predict the partitioning of small molecules into nucleolar condensates in living cells. Different biomolecular condensates harbor distinct chemical environments, that the chemical grammar of condensates can be ascertained by machine learning.

### RELATED APPLICATIONS

This application claims the benefit of U.S. Provisional Application No. 63/363,572, filed on Apr. 25, 2022 and U.S. Provisional Application No. 63/476,084, filed on Dec. 19, 2022. The entire teachings of the above applications are incorporated herein by reference.

### GOVERNMENT SUPPORT

This invention was made with government support under Grant No. GM123511 from the National Institutes of Health. This invention was made with government support under Grant No. CA155258 from the National Institutes of Health. This invention was made with government support under Grant No. PHY2044895 from the National Science Foundation. The government has certain rights in the invention.

### BACKGROUND

A wide array of cellular functions—including DNA replication and repair, transcription, splicing, signaling, and ribosome biosynthesis—have been reported to occur in biomolecular condensates (1-7). The insides of condensates have been proposed to possess distinct chemical environments that are densely concentrated with certain proteins and nucleic acids that together solvate and enrich specific sets of biomolecules (6). The internal environments of condensates have physicochemical properties that can influence biomolecular activity (9, 10), consistent with the notion that these environments differ from the external milieu. These solvation environments are produced by the ensemble of components within a condensate, as opposed to the local chemical environment produced by a segment of a structured protein where a small molecule has a single high-affinity binding site (8). The condensates characterized to date differ in their molecular composition and function and may thus have different solvation environments, but there is limited evidence for such differences (1-7). Although protein and RNA molecules have been shown to selectively partition into certain condensates, it is possible that this selectivity emerges from direct interactions with other biomolecules within the condensate rather than the solvation environment intrinsic to each condensate.

### SUMMARY

The method described herein involve training a machine-learning classifier on in vitro data to predict outcomes in vivo. The particular application of the technique described herein involves a computer-implemented method of quantifying partitioning of one or more test agents in an in vivo condensate based on a training dataset. The training dataset includes data pertaining to quantification of partitioning of training agents in an in vitro protein condensate that corresponds to the in vivo condensate. The training dataset also includes a representation of training agents (e.g., computer-readable information regarding the agents, such as chemical structure and/or chemical properties of the agents).

Described herein is a computer-implemented method of quantifying partitioning of one or more test agents in an in vivo condensate. The method includes training a machine-learning classifier on a training dataset, the training dataset comprising (i) a quantification of partitioning of training agents in an in vitro protein condensate that corresponds to the in vivo condensate and (ii) a representation of the training agents; and applying a test dataset comprising a representation of the one or more test agents to the machine-learning classifier to quantify partitioning of the one or more test agents in the in vivo condensate.

Described herein is a method of quantifying partitioning of one or more test agents in an in vivo condensate. The method can include: applying a test dataset comprising a representation of the one or more test agents to a machine-learning classifier to quantify partitioning of the one or more test agents in the in vivo condensate, the machine-learning classifier trained on a training dataset that comprises (i) a quantification of partitioning of training agents in an in vitro protein condensate that corresponds to the in vivo condensate and (ii) a representation of one or more training agents. The machine learning algorithm can be a random forest classifier. The machine learning algorithm can be a message-passing neural network.

Described herein is a system for quantifying partitioning of one or more test agents in an in vivo condensate. The system includes: a processor; and a memory with computer code instructions stored thereon, the processor and the memory, with the computer code instructions, being configured to cause the system to: train a machine-learning classifier on a training dataset, the training dataset comprising (i) a quantification of partitioning of training agents in an in vitro protein condensate that corresponds to the in vivo condensate and (ii) a representation of the training agents; and apply a test dataset comprising a representation of the one or more test agents to the machine-learning classifier to quantify partitioning of the one or more test agents in the in vivo condensate.

Described herein is a non-transitory computer readable medium with instructions stored thereon for quantifying partitioning of one or more test agents in an in vivo condensate. The instructions, when executed by a processor, cause the processor to: train a machine-learning classifier on a training dataset, the training dataset comprising (i) a quantification of partitioning of training agents in an in vitro protein condensate that corresponds to the in vivo condensate and (ii) a representation of the training agents; and apply a test dataset comprising a representation of the one or more test agents to the machine-learning classifier to quantify partitioning of the one or more test agents in the in vivo condensate.

Described herein is a system for quantifying partitioning of one or more test agents in an in vivo condensate. The system includes: a processor; and a memory with computer code instructions stored thereon, the processor and the memory, with the computer code instructions, being configured to cause the system to: apply a representation of the one or more test agents to a machine-learning classifier trained on a training dataset that comprises (i) a quantification of partitioning of training agents in an in vitro protein condensate that corresponds to the in vivo condensate and (ii) a representation of the training agents; and quantify a partitioning of the one or more test agents in the in vivo condensate.

Described herein is a non-transitory computer readable medium with instructions stored thereon for quantifying partitioning of one or more test agents in an in vivo condensate, the instructions, when executed by a processor, causing the processor to: apply a representation of the one or more test agents to a machine-learning classifier trained on a training dataset that comprises (i) a quantification of partitioning of training agents in an in vitro protein condensate that corresponds to the in vivo condensate and (ii) a representation of the training agents; and quantify a partitioning of the one or more test agents in the in vivo condensate.

Embodiments of the methods, systems, and non-transitory computer readable media can each include several features:

The machine-learning classifier can be a random forest classifier. The machine-learning classifier can be a message passing neural network. The message-passing neural network can be a directed message-passing neural network.

Training the machine-learning classifier can further include training a first machine-learning classifier on the training dataset, and training a second machine-learning classifier on the training dataset. Applying the test dataset that includes the representation of the one or more test agents to the machine learning-classifier can further include applying the test dataset that includes the representation of the one or more test agents to the first machine-learning classifier and the second machine-learning classifier, thereby producing results from each respectively. Embodiments can further include aggregating the respective results of the first machine-learning classifier and the second machine-learning classifier to quantify partitioning of the one or more test agents in the in vivo condensate.

Aggregating the respective results can include determining whether the result of the first machine-learning classifier and the second machine-learning classifier indicate that a partitioning ratio of the one or more test agents exceed specified probability thresholds for the first machine-learning classifier and the second machine-learning classifier; and if both of the respective results exceed the specified probability thresholds, quantifying the partitioning of the one or more test agents in the in vivo condensate based on the partitioning ratio.

The machine-learning classifier can be one or more of a neural network, an artificial neural network, a graph neural network, a sequence neural network, a binary classifier, a forest classifier, a random forest classifier, and a message passing neural network.

The training dataset can be provided.

The quantification of partitioning of training agents in the in vitro protein condensate can be a partition ratio of a quantification of the training agents within the in vitro protein condensate versus a quantification of the training agents outside the in vitro protein condensate.

Training the message-passing neural network can include associating the representation of the training agents with one or more partition ratios in one or more condensates.

The representations of the one or more test agents and training agents can be a representation of chemical structure. The representation of the one or more test agents and training agents can be a simplified molecular-input line-entry system (SMILES) representation of chemical structure. The representation of the one or more test agents and training agents can be a Morgan fingerprint of chemical structure. The representation of the one or more test agents and training agents can include chemical properties. The chemical properties can be a vector comprising chemical property data.

Embodiments can include selecting a threshold for solvation, wherein the quantified partitioning of the one or more test agents in the in vivo condensate above the threshold indicates that the one or more test agents solvate in the in vivo condensate.

Embodiments can include applying a validation dataset that includes a representation of one or more validation agents to the machine-learning classifier.

Embodiments can include comparing a quantified partitioning of the one or more test agents in a first in vivo condensate to a quantified partitioning of the one or more test agents in a second in vivo condensate.

The in vitro protein condensate can include a condensate selected from Table 1. The in vivo protein condensate can include a condensate selected from Table 1. The in vitro protein condensate can include MED1. The in vitro protein condensate can include NPM1. The in vitro protein condensate can include HP1α. The in vivo protein condensate can include MED1. The in vivo protein condensate can include NPM1. The in vivo protein condensate can include HP1α.

The one or more test agents can include at least one of a small molecule, an RNA, an siRNA, a peptide, and a candidate therapeutic agent.

Embodiments can include selecting a test agent based on the quantified partitioning of the test agent in the in vivo condensate. The quantified partitioning of the selected test agent in the in vivo condensate can be greater than or equal to a selected threshold for solvation. The quantified partitioning of the selected test agent in the in vivo condensate can be less than or equal to a selected threshold for solvation. Embodiments can include administering the selected test agent to cells to determine in vivo partitioning of the test agent.

Embodiments can include repeating a) and b) for a plurality of in vitro protein condensates for a corresponding plurality of in vivo condensates. Embodiments can include comparing the quantified partitioning of the one or more test agents in the plurality of in vivo condensates.

Embodiments can include selecting a test agent based on relative partitioning of the test agent into the plurality of in vivo condensates. Embodiments can include administering the selected test agent to cells to determine in vivo partitioning of the selected test agent into the plurality of in vivo condensates.

The in vivo condensate can include a biological target of the selected test agent.

Embodiments can include generating the training dataset by: forming an in vitro condensate of a protein; administering training agents to the condensate; detecting a signal inside the condensate and signal outside the condensate; determining a partition ratio of the signal inside the condensate divided by the signal outside the condensate; and repeating a) through d) for a plurality of training agents to generate the training dataset. The protein of the in vitro condensate can be fused to a tag The tag can be a fluorescent protein, and detecting the signal can include detecting a fluorescent signal.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1. Therapeutic small molecule drugs concentrate in distinct intracellular environments. Micrographs showing live HCT-116 cells that were incubated with endogenously fluorescent drugs (50 μM) for 1 hour and imaged with a confocal microscope. Dashed-line boxes indicate the origin of each zoom (2×) cutout source, scale bar: 10 μm. R=(Thr-D-Val-Pro-Sar-MeVal), R₁=p-chlorobenzene, R₂=CH₂CH₂OCH₂CH₂NH₂, R₃═CH₂CH₂N(CH₂CH₂)₂.

FIGS. 2A-H. Selective partitioning of small molecules in simple condensates (FIG. 2A) Top: Images of condensates in HCT-116 cells expressing MED1-GFP (transcriptional condensates), NPM1-GFP (nucleolar condensates) and HP1α-GFP (heterochromatin condensates). Bottom: Scaffold proteins above were engineered as BFP fusion proteins forming in vitro condensates to measure probe partitioning (Top scale bar: 10 μm, 2.0× zoom, Bottom scale bar: 2 μm). (FIG. 2B) Chemical scaffolds of fluorescent probes used to measure partitioning within condensate assays and example R-groups. (FIG. 2C) Schematic of in vitro condensate partitioning screen and calculation of the partition ratio, K (FIG. 2D) 3-D scatter plot of fluorescent probes compared across condensates; color gradient is proportional to MED1 partition ratio. Blue, red, and green dots correspond to probes in FIG. 2E. (FIG. 2E) Chemical structures of the highest partitioning probe for each condensate, compared by partition ratio in other condensates. Blue, red, and green dots reference points in FIG. 2D. (FIGS. 2F-H) Dot plots showing the percentile rank partitioning of probes into condensates compared to their partitioning into others (FIG. 2F) MED1, (FIG. 2G) NPM1, and (FIG. 2H) HP1α.

FIGS. 3A-F. Probe chemical features suggest a chemical grammar in condensates. (FIG. 3A) Cartoon depicting how similar molecules (here, sharing color) might interact with the same chemical environment (FIG. 3B) Schematic showing calculation of Tanimoto similarity matrices comparing fluorescent probes by their Morgan Fingerprint's. (FIG. 3C) Schematic and (FIG. 3D) dot plots showing calculation of mean Tanimoto similarities from matrices fluorescent probes compared against each other in high-to-high (H-H), high-to-low (H-L) and low-to-low (L-L) partitioning regions. (FIG. 3E) Graphic and (FIG. 3F) dot plots show the comparison of high partitioning probes between correlates through quantification of matrices, p=****. (p-value, p. ****p<0.0001, ***0.0001<p<0.001, **0.001<p<0.01, * 0.05<p<0.01).

FIGS. 4A-H. Deep learning discovers compounds with selective partitioning behaviors (FIG. 4A) Schematic of message passing neural network for classifying probe partitioning behaviors into in vitro condensates. (FIG. 4B) Bar graph showing the median partition ratio of deep learning (DL) and randomly selected probes (RS). (FIGS. 4C-E) Cumulative distribution function of fluorescent probes selected by DL or RS in (FIG. 4C) MED1, (FIG. 4D) NPM1 and (FIG. 4E) HP1a in vitro droplet assays. (FIG. 4F) Bar graph depicting the efficiency of selecting probes above a condensate's partition ratio threshold with DL or RS. (FIG. 4G) Bar graph depicting the precision of deep learning models generated for each condensate. (FIG. 4H) Cumulative distribution function showing the Tanimoto similarity of the DL selected fluorescent probes between each of the condensates considered.

FIGS. 5A-B. Live cell partitioning predicted by deep learning classifiers. (FIG. 5A) Live cell confocal images of HCT-116 cells incubated with drugs (magenta) classified by the NPM1 deep learning (DL) classifier and quantification, (N.D.=not determined). Ratio of signal inside the nucleolus and outside the nucleolus is shown on the right. Analysis of NPM1 model predictions (FIG. 15) provided the following metrics: accuracy (ACC)=0.63, balanced accuracy (BA)=0.57, F1=0.40, an informed-ness (I)=0.13, and DOR=2 (95% CI, 0.46-8.55), (see supporting information). (FIG. 5B) Live cell confocal images of mouse embryonic stem cells (mESCs) incubated with Hoechst stain (green) and the drugs selected by the HP1α DL classifier (magenta) concentrate in mESC chromocenters. Quantification of the ratio of signals inside each chromocenter compared to the outside is shown on the right. Analysis of HP1α model predictions (FIG. 15) provided the following metrics: accuracy (ACC)=0.95, balanced accuracy (BA)=0.86, F1=0.75, an informed-ness (I)=0.72, and DOR=105 (95% CI, 5-2, 135). Scale bars: 10 μm.

FIG. 6. Live cell confocal and Two-photon imaging of endogenously fluorescent drugs. Cells were incubated with a drug or natural product at 50 μM for 1 hour and then imaged with a confocal or two-photon microscope Scale: 10 μm.

FIG. 7. Live cell Two-photon imaging of FDA drug and natural products in cancer cells. Live HCT-116 cells were incubated with a drug or natural product at 50 μM for 1 hour prior to two-photon imaging. Drugs and natural products are listed in FIG. 14. Scale: 10 μm.

FIGS. 8A-B. 3-D scatter plot of fluorescent probes compared across each condensate. (FIG. 8A) NPM1 partition ratio (red to black), (FIG. 8B) HP1α partition ratio (green to black). Color gradient is dictated by the probes partition ratio in NPM1 (FIG. 8A) and (FIG. 8B) HP1α respectively.

FIGS. 9A-B. All data collected in in vitro droplet assay. (FIG. 9A) Dot plot showing the distribution of partition ratios of fluorescent probes in MED1, NPM1, and HP1α condensates and the partition ratio mean and variance. (FIG. 9B) Dot plot showing the same data as in (FIG. 9A), but on the range of [0, 1.5].

FIGS. 10A-I. Additional analysis of condensate selectivity in fluorescent probe partitioning. (FIGS. 10A-C) Dot plots showing the 90^{th} percentile partitioning probes compared to those probes in other condensates. (FIGS. 10D-F) Dot plots comparing the partition ratios of probes with partition ratios, 1.30≥K≥0.90, in (FIG. 10D) MED1, (FIG. 10E) NPM1, and (FIG. 10F) HP1α against their percentiles in other condensates. (FIGS. 10G-I) Dot plots comparing the 10th percentile of probes in (FIG. 10G) MED1, (FIG. 10H) NPM1, and (FIG. 10I) HP1α against their percentiles in other condensates. (p-value, p. **** p<0.0001, ***0.0001<p<0.001, **0.001<p<0.01, * 0.05<p<0.01).

FIGS. 11A-G. Tanimoto similarity matrices and comparison marices. Matrices were rank ordered (from high partitioning, red, to low partitioning, white) Tanimoto similarity matrices, quantified in FIG. 2D (FIG. 11A) MED1, (FIG. 11B) NPM1, and (FIG. 11C) HP1α. Darker blue indicates more similar molecules, white indicates molecules with none or few shared features. Side-bar red color gradient indicates increasing partition ratio. (FIG. 11D) High partitioning probes (90th percentile or above) are compared in the bottom left hand and top right-hand corner for each condensate pair. Comparison matrices quantified in FIG. 2F, for (FIG. 11E) MED1 and NPM1, (FIG. 11F) MED1 and HP1α, and (FIG. 11G) NPM1 and HP1α.

FIG. 12. Live cell Two-photon imaging of FDA drug and natural products in mouse embryonic stem cells. Live mouse embryonic stem cells were incubated with a drug or natural product at 50 μM for 1 hour prior to two-photon imaging. Drugs and natural products are listed in FIG. 14. Scale: 50 μm.

FIG. 13. Confocal imaging of FDA drugs and natural products in mouse embryonic stem cells. Live mouse embryonic stem cells were incubated with a drug or natural product at 50 μM for 1 hour prior to confocal imaging. Drugs and natural products are listed in FIG. 14. Scale: 10 μm.

FIG. 14 is a table of the subcellular distribution of endogenously fluorescent FDA drugs and natural products.

FIG. 15 is a table of nucleolar and chromocenter enrichment compared against the NPM1 and HP1α deep learning classifier prediction of FDA drugs and natural products.

FIG. 16 illustrates a computer network or similar digital processing environment in which embodiments of the present invention may be implemented.

FIG. 17 is a diagram of an example internal structure of a computer (e.g., client processor/device 50 or server computers 60) in the computer system of FIG. 16.

### DETAILED DESCRIPTION

A description of example embodiments follows.

### Introduction

A wide array of cellular functions—including DNA replication and repair, transcription, splicing, signaling, and ribosome biosynthesis—have been reported to occur in biomolecular condensates (1-7). The insides of condensates have been proposed to possess distinct chemical environments that are densely concentrated with certain proteins and nucleic acids that together solvate and enrich specific sets of biomolecules (6, 8). The internal environments of condensates have physicochemical properties that can influence biomolecular activity (9, 10), consistent with the notion that these environments differ from the external milieu. These solvation environments are produced by the ensemble of components within a condensate, as opposed to the local chemical environment produced by a segment of a structured protein where a small molecule has a single high-affinity binding site (8). The condensates characterized to date differ in their molecular composition and function and may thus have different solvation environments, but there is limited evidence for such differences (1-7). Although protein and RNA molecules have been shown to selectively partition into certain condensates, it is possible that this selectivity emerges from direct interactions with other biomolecules within the condensate rather than the solvation environment intrinsic to each condensate.

We have shown that certain anticancer drugs can concentrate in specific biomolecular condensates and do so by mechanisms that are independent of target binding (11), which is consistent with the possibility that some condensates create a specific solvation environment for certain small molecules that differs from that outside the condensate. A more thorough understanding of the internal solvation properties of biomolecular condensates is needed to address whether the chemical environments of specific condensates are distinct, can contribute to selective partitioning of small molecules, and might be useful to improve the pharmacological activity of therapeutics (8, 12). Current approaches to drug discovery do not yet account for the impact of biomolecular condensates on the subcellular distribution of small molecules, in part because it is not clear whether there are chemical rules that govern selective partitioning of such molecules in condensates.

Here, we show that small molecule drugs concentrate in distinct intracellular environments, some bounded by membranes and others that are non-membrane containing condensates. We used a library of fluorescent small molecule probes to investigate the local chemical environments of biomolecular condensates in vitro. We found that different protein condensates formed in vitro possess distinct chemical solvation properties, that the chemical rules that govern selective partitioning of small molecules in these condensates can be ascertained by deep learning, and that these rules predict the condensate partitioning behavior of small molecules. The partitioning rules ascertained with simple protein condensates in vitro correctly predicted that some drugs would selectively concentrate in the more complex environment of nucleolar condensates in cells, although the quality of these predictions was considerably less than that for the simpler condensates formed in vitro. Our results show that different biomolecular condensates possess distinct chemical solvating environments, indicate that there are chemical rules that govern selective partitioning and determine the subcellular distribution of small molecules, and suggest that further discovery of these rules may facilitate development of small molecule therapeutics with optimal subcellular distribution and therapeutic benefit.

### Overview of Machine Learning

Most machine learning involves transforming data in some sense. A machine learning model can be a computational machinery for ingesting data of one type, and outputting predictions of a possibly different type. For example, statistical models can be estimated from input data. Deep learning is differentiated from classical approaches principally by the set of powerful models that it focuses on. These models consist of many successive transformations of the data that are chained together top to bottom (e.g., in layers or dimensions), thus the name deep learning.

A random forest classifier is an ensemble learning method that constructs a multitude of decision trees during training. The output of the random forest is the class selected by most trees.

Embodiments described herein refer to a directed message-passing neural network. An undirected message-passing neural network can also be used, but prior work has shown that directed message-passing neural networks can achieve better results due to the inductive bias they introduce to the model. Yang et al., Analyzing Learned Molecular Representations for Property Prediction, J. Chem. Inf. Model. 2019, 59, 8, 3370-3388. In some embodiments, the neural network can be a graph-based neural network. In some embodiments, the neural network can be a sequence-based neural network.

### Application of Machine Learning to Protein Condensates

The agents can be a variety of different types of agents, such as small molecules, RNA, siRNA, peptides, and proteins.

Preferably, the agents of the training dataset exhibit a variety of chemical characteristics, such as a range of hydrophobicity, lipophilicity, aromaticity, acid-base, pKa, and molecular weight, to name a few. In general, larger training datasets are preferable to smaller training datasets, but one should avoid overtraining by using training agents having too little dissimilarity, which can introduce bias into the machine learning system. With the foregoing in mind, it is typically unnecessary for the training dataset to include agents that are vastly different from the agents of interest of the test dataset. In some embodiments, the training dataset includes at least 100 training agents. In some embodiments, the training dataset includes at least 500 training agents. In some embodiments, the training dataset includes at least 1000 training agents. In some embodiments, the training dataset includes at least 5,000 training agents. In some embodiments, the training dataset includes at least 10,000 training agents.

In some embodiments, the representation of the one or more test agents and training agents describes chemical structure of the one or more test agents and training agents. One example is a simplified molecular-input line-entry system (SMILES) representation of the agents. Another example is a Morgan fingerprint. Another example is chemical property information, such as Chemprop uses the RDKit package to also transform

In the embodiments described here, two machine learning classifiers were used. The random forest classifier and the directed message-passing neural network described herein are complementary in nature in terms of how they operate. Larger training datasets can allow for improved accuracy with a single machine-learning classifier. Among the two embodiments described herein, the directed message-passing neural network is a preferred embodiment.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 Daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, the small molecule comprises at least one, at least two, at least three, or more aromatic side chains.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc. In some embodiments, the agent is a protein or polypeptide comprising at least 5, at least 10, at least 15, at least 20, at least 25, at least 30, or more aromatic amino acids.

In some embodiments, the agent consists of or comprises DNA or RNA.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

The agent may be a known drug. The type of drug is not limited any may be any suitable drug. In some embodiments, the agent may be an anti-cancer drug. In some embodiments, the known drug is to treat a human disease or condition.

In some embodiments, the agent is a chemotherapeutic or a derivative thereof. In some embodiments, the chemotherapeutic agent is selected from actinomycin D, aldesleukin, alitretinoin, all-trans retinoic acid/ATRA, altretamine, amascrine, asparaginase, azacitidine, azathioprine, bacillus calmette-guerin/BCG, bendamustine hydrochloride, bexarotene, bicalutamide, bleomycin, bortezomib, busulfan, capecitabine, carboplatin, carfilzomib, carmustine, chlorambucil, cisplatin/cisplatinum, cladribine, cyclophosphamide/cytophosphane, cytabarine, dacarbazine, daunombicin/daunomycin, denileukin diftitox, dexrazoxane, docetaxel, doxorubicin, epimbicin, etoposide, fludarabine, fluorouracil (5-FU), gemcitabine, goserelin, hydrocortisone, hydroxyurea, idambicin, ifosfamide, interferon alfa, irinotecan CPT-11, lapatinib, lenalidomide, leuprolide, mechlorethamine/chlormethine/mustine/HN2, mercaptopurine, methotrexate, methylprednisolone, mitomycin, mitotane, mitoxantrone, octreotide, oprelvekin, oxaliplatin, paclitaxel, pamidronate, pegaspargase, pegfilgrastim, PEG interferon, pemetrexed, pentostatin, phenylalanine mustard, plicamycin/mithramycin, prednisone, prednisolone, procarbazine, raloxifene, romiplostim, sargramostim, streptozocin, tamoxifen, temozolomide, temsirolimus, teniposide, thalidomide, thioguanine, thiophosphoamide/thiotepa, thiotepa, topotecan hydrochloride, toremifene, tretinoin, valmbicin, vinblastine, vincristine, vindesine, vinorelbine, vorinostat, zoledronic acid, and combinations thereof. In some embodiments, the agent is or comprises cisplatin or a derivative thereof. In some embodiments, the agent is or comprises JQ1 ((S)-tert-butyl 2-(4-(4-chlorophenyl)-2,3,9-trimethyl-6H-thieno[3,2-/][1, 2,4]triazolo [4,3-a [1,4]diazepin-6-yl)acetate) or a derivative thereof. In some embodiments, the agent is or comprises tamoxifen or a derivative thereof.

In some embodiments, the agent comprises a protein transduction domain (PTD). A PTD or cell penetrating peptide (CPP) is a peptide or pep to id that can traverse the plasma membrane of many, if not all, mammalian cells. A PTD can enhance uptake of a moiety to which it is attached or in which it is present. Often such peptides are rich in arginine. For example, the PTD of the Tat protein of human immunodeficiency viruses types 1 and 2 (HIV-1 and HIV-2) has been widely studied and used to transport cargoes into mammalian cells. See, e.g., Fonseca S B, et ah, Adv Drug Deliv Rev., 61(11): 953-64, 2009; Heitz F, et ah, Br J Pharmacol., 157 (2): 195-206, 2009, and references in either of the foregoing, which are incorporated herein by reference. In some embodiments, the cell penetrating peptide is HIV-TAT.

In some embodiments, the agent is capable of binding to a target. In some embodiments, the target is present in the composition comprising the condensate. In some embodiments, the target is predominantly present (e.g., at least 51%, at least 60%, at least 70%, at least 80%, at least 90%, at least 95%, at least 97%, at least 99%, at least 99.5%, at least 99.9%, at least 99.99%, or more) outside of the condensate. In some embodiments, the concentration of the target outside of the condensate is at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold, or more than the concentration of the target inside the condensate. In some embodiments, the target is predominantly present (e.g., at least 51%, at least 60%, at least 70%, at least 80%, at least 90%, at least 95%, at least 97%, at least 99%, at least 99.5%, at least 99.9%, at least 99.99%, or more) in the condensate. In some embodiments, the concentration of the target in the condensate is at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold, or more than the concentration of the target outside the condensate.

In some embodiments, the agent is a candidate agent as described herein. In some embodiments, the agent is resultant from an agent has been modified to modulate incorporation into a condensate of interest. In some embodiments, the agent is resultant from the coupling or linking of a first agent and second agent as described herein.

### Computer Implementation

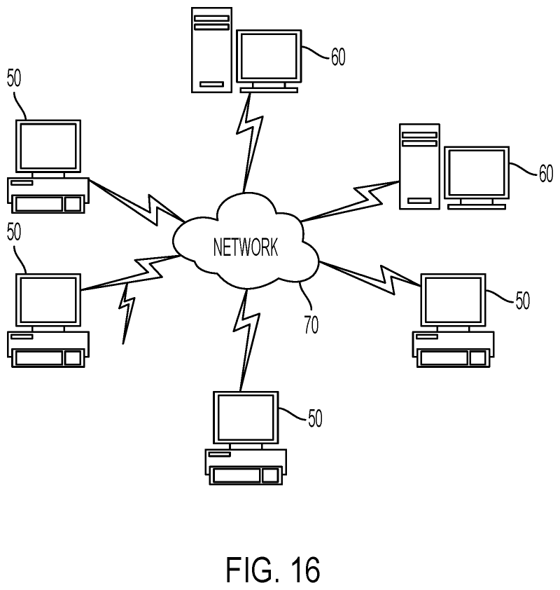

FIG. 16 illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

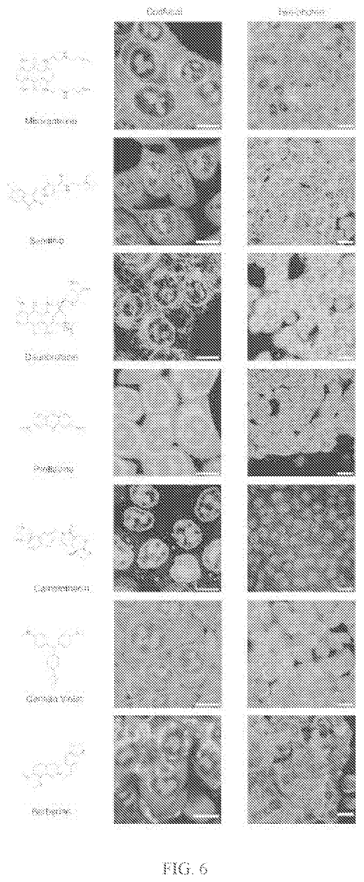

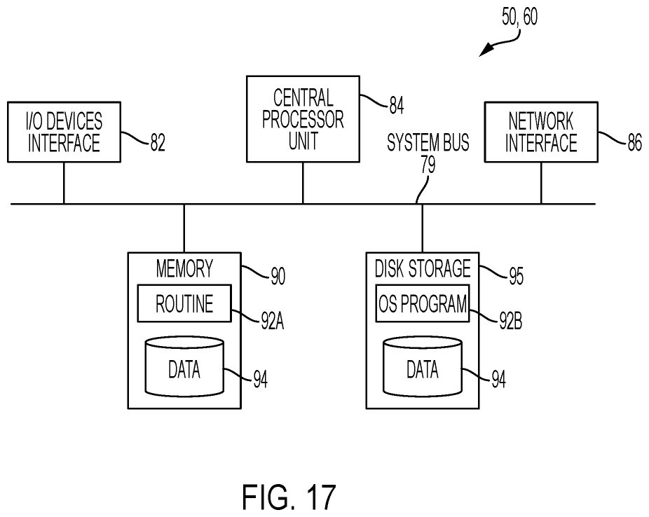

FIG. 17 is a diagram of an example internal structure of a computer (e.g., client processor/device 50 or server computers 60) in the computer system of FIG. 16. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement an embodiment of the present invention (e.g., random tree forest classifier module, MPNN module code detailed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

### Therapeutic Small Molecule Drugs Concentrate in Distinct Intracellular Environments

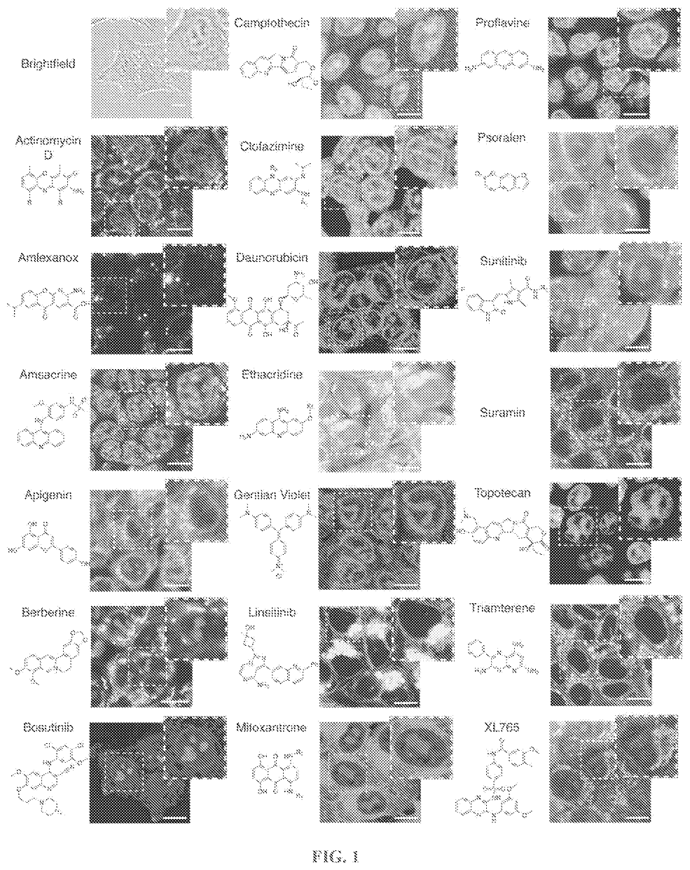

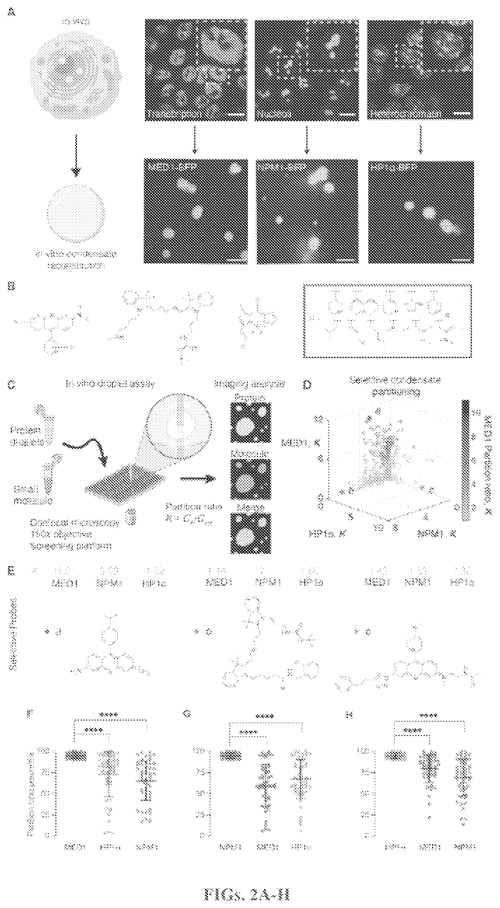

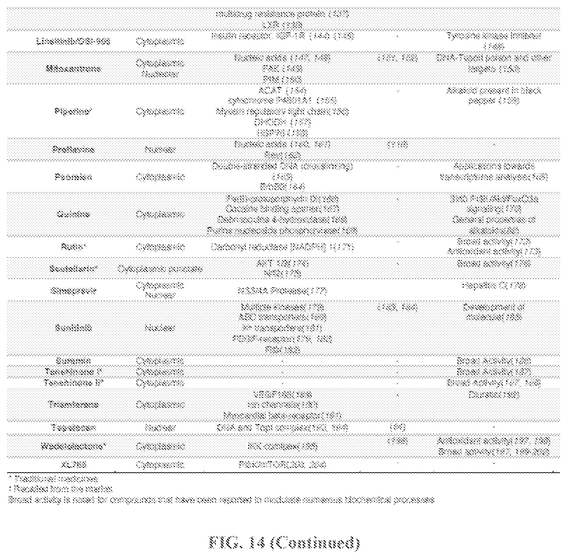

Previous studies have noted that certain small molecules will distribute in a discontinuous fashion throughout cells, apparently concentrating in subcellular compartments (13-41). These observations were made with different compounds in diverse cells under varying conditions. To provide a more systematic investigation of the intracellular distribution of a collection of therapeutic small molecules in a single cell type under identical conditions, we selected a set of twenty drugs whose structures indicate they are endogenously fluorescent, including FDA-approved drugs and natural products, and imaged their distribution in live HCT-116 cells with confocal microscopy. The distribution of fluorescent signal for all small molecules was discontinuous in these cells and showed various spatial patterns (FIG. 1, FIG. 14), suggesting that many therapeutically important small molecule compounds become distributed in distinct subcellular environments.

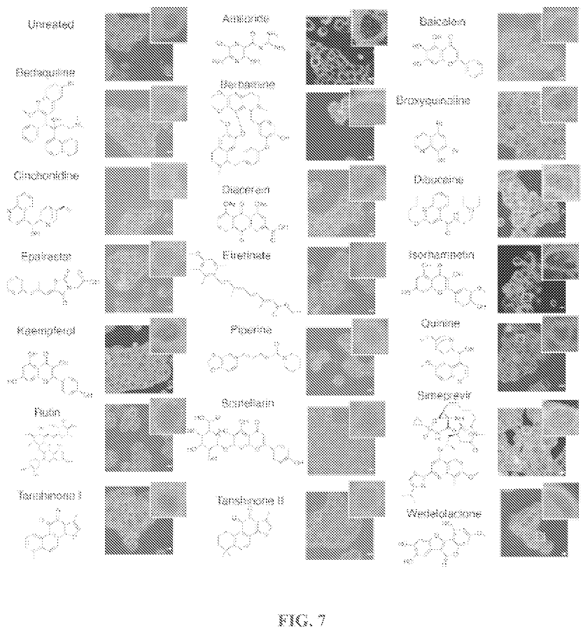

Few drugs are endogenously fluorescent in the range of visible light, so we developed a two-photon imaging assay to interrogate the subcellular distribution in live cells of additional small molecules likely to possess a fluorescent excitation peak in the ultraviolet region. For a subset of the small molecules that were studied with confocal imaging, we confirmed that the two-photon imaging assay revealed the same discontinuous pattern of cellular distribution, although the images produced in this assay are lower resolution (FIG. 6). We then used two-photon imaging with twenty additional compounds, which included quinine, dibucaine, simeprevir, various quinoline drugs and a variety of natural products, again observing that most of these compounds exhibited a discontinuous distribution is cells (FIG. 7, FIG. 14).

Some patterns of signal from the small molecules were concentrated in organelles with well-recognized features (FIG. 14). For example, fluorescent signals for the drugs camptothecin, proflavine, sunitinib, and topotecan were concentrated almost exclusively in the nucleus, whereas those for amlexanox, linsitinib, suramin, and triamterene were concentrated predominantly in the cytoplasm. The nucleolus, a well-studied condensate, appeared to concentrate sunitnib and mitoxantrone, among others. Berberine found concentrated predominantly in mitochondria.

Notably, some of the drugs studied here concentrated in compartments where their established high-affinity targets occur, but others did not. For example, topotecan is a topoisomerase inhibitor and much of its fluorescent signal occurred in the nucleus where its target resides. In contrast, sunitinib and bosutinib are anti-cancer tyrosine kinase inhibitors whose targets are thought to reside in the lipid bilayer and perhaps the cytoplasm, but much of the signal for these drugs was concentrated in the nucleoli. These results indicate that some small molecule therapeutics are concentrated in subcellular compartments where they readily access their targets, as we have noted previously for cisplatin and tamoxifen, which concentrate in transcriptional condensates (11). However, some drugs appeared to be distributed to subcellular compartments that lack their targets, and thus their distribution may not be optimal for target engagement and might instead produce toxic effects by engaging unintended targets.

### Selective Partitioning of Small Molecule Probes in Simple Biomolecular Condensates

The observation that many drugs concentrate in subcellular compartments, coupled with recent evidence that many cellular functions are compartmentalized in biomolecular condensates, compelled us to investigate whether condensates harbor distinct chemical environments that might account for the selective concentration of small molecule drugs. A chemical solvation environment within a biological system is a product of the complementary solvation properties of proteins, water, metabolites, ions, and other macromolecules. Differences between the chemical solvation environment inside and outside of a condensate would be anticipated to cause a small molecule to differentially partition between the condensate and the external milieu (8). The degree of small molecule partitioning is dictated by the respective solvation properties of each phase and the physicochemical properties of the small molecule under investigation.

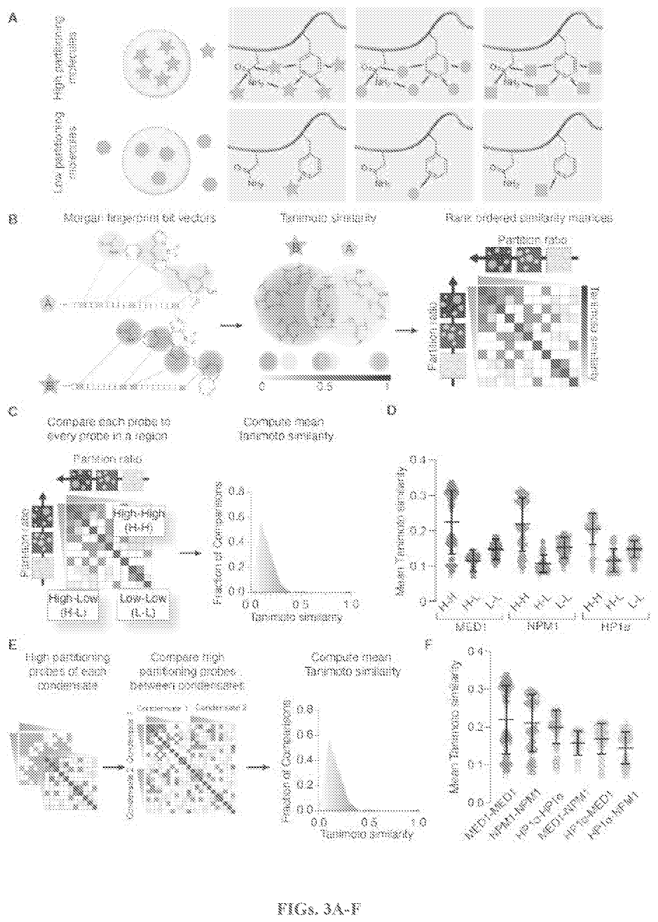

Biomolecular condensates contain many different proteins, yet some proteins appear to play dominate roles due to frequent interactions with other proteins and perhaps relative abundance; these “scaffold” proteins have been purified and used to create homotypic condensates in vitro that permit analysis of condensate properties (13-15). We used the scaffold proteins of transcriptional (MED1), nucleolar (NPM1) and heterochromatic (HP1α) condensates, fused to blue fluorescent protein, to produce homotypic condensates appropriate for small molecule screening in vitro (FIG. 2A). A library of fluorescent probes with a variety of chemical structures was used to test differential partitioning. The probes in this library consisted of xanthene, boron dipyrromethene (BODIPY) or cyanine fluorophore scaffolds chemically derivatized with up to three different R-groups, sampling combinations of various aromatic, heteroaromatic, aliphatic, basic, acidic, carbonyl, and halogenated moieties (FIG. 2B). In total, over 1,500 chemical probes were used to test for chemical features that might cause small molecules to partition selectively into MED1, NPM1 and HP1α condensates. A 384-well plate confocal imaging assay was used to measure the partition ratio (K) of each of the chemical probes, where K was defined as the ratio of fluorescent signal intensity inside versus outside the droplets (FIG. 2C).

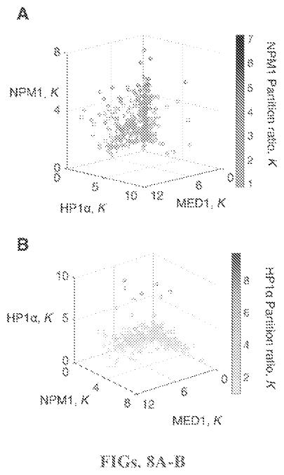

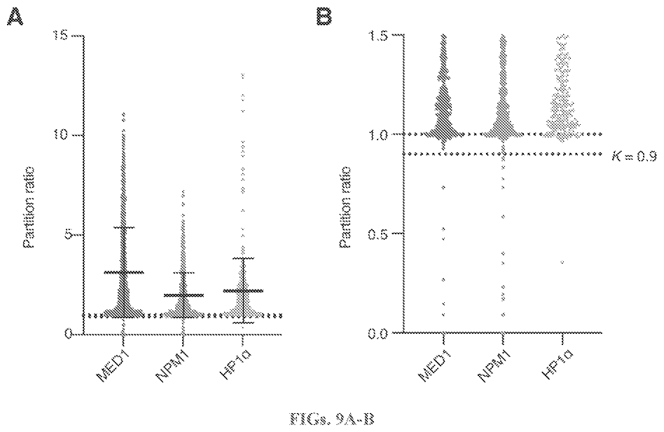

The results of the small molecule screen indicated that all chemical probes were capable of diffusing into the droplets and that many probes were enriched in one or more condensates (FIGS. 2D, and 8A-B). A substantial portion of the probes exhibited partition ratios of at least 2-fold greater than passive diffusion (52% for MED1, 33% for NPM1 and 27% for HP1α condensates). Some probes initially appeared to be somewhat excluded from one or more of the condensates (K<0.9), but further analysis revealed that this was not due to probe exclusion but rather experimental or analytical artifacts, so these probes were omitted from the analysis (FIGS. 9A-B). The three molecules that were the highest partitioning probes in the three condensates are shown in FIG. 2E; each of these showed only modest partitioning in the other two condensates, suggesting that the concentrating effect was specific to each condensate.

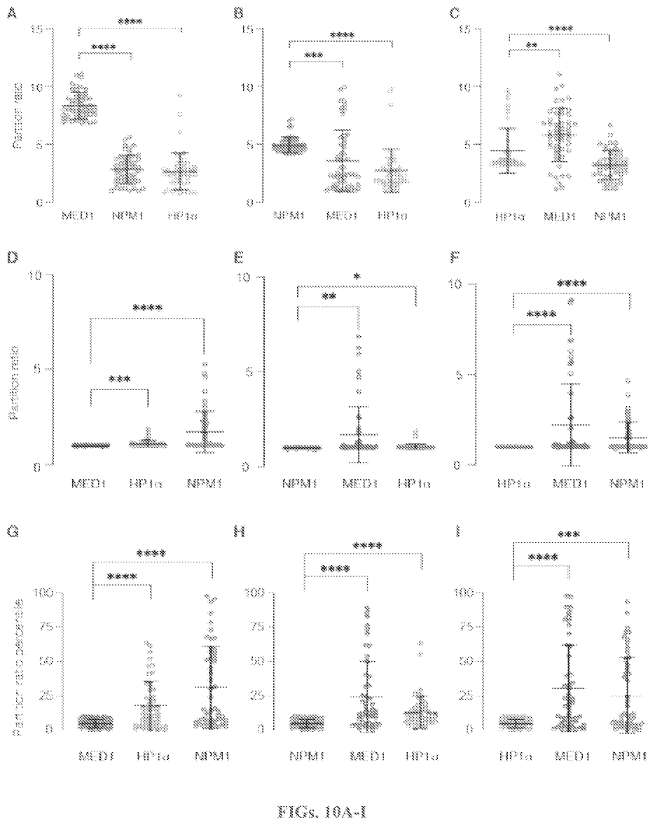

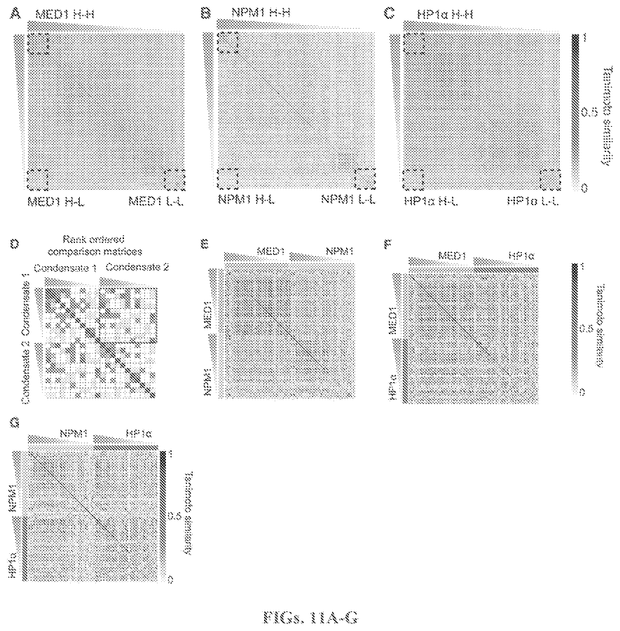

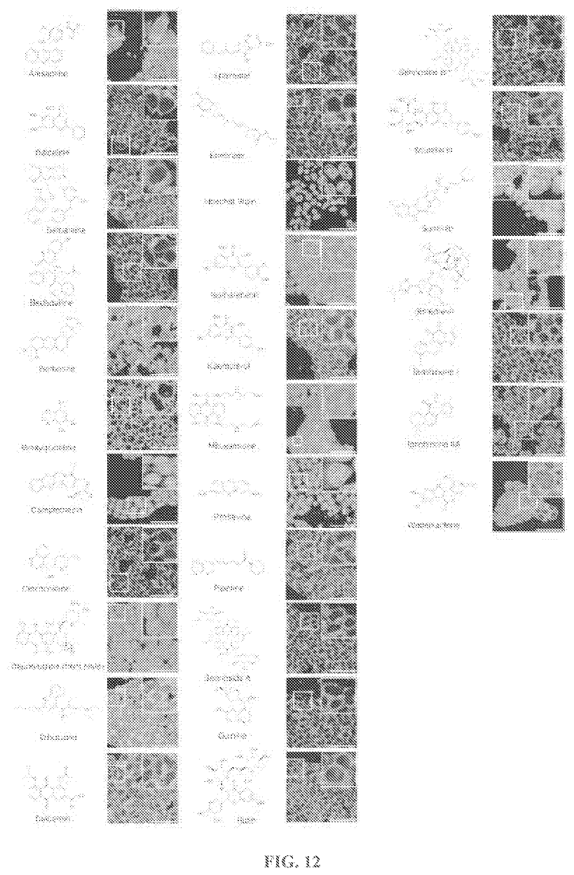

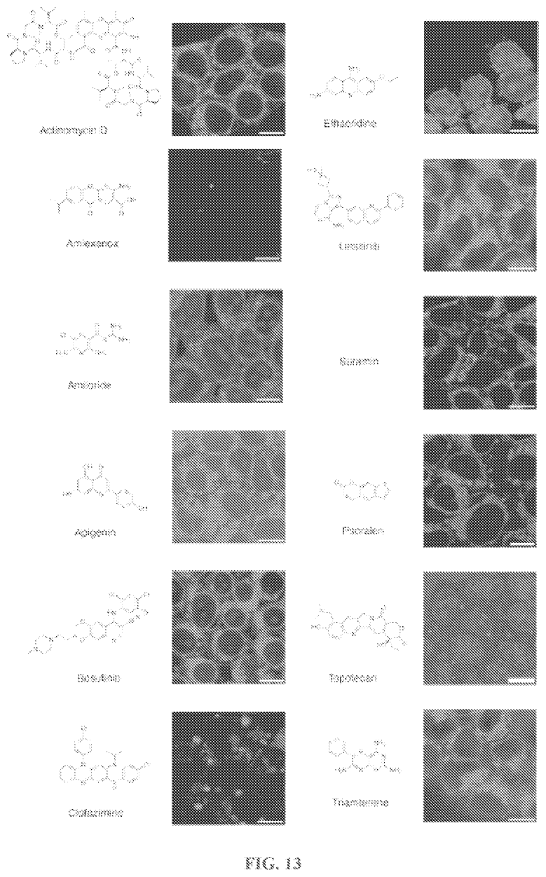

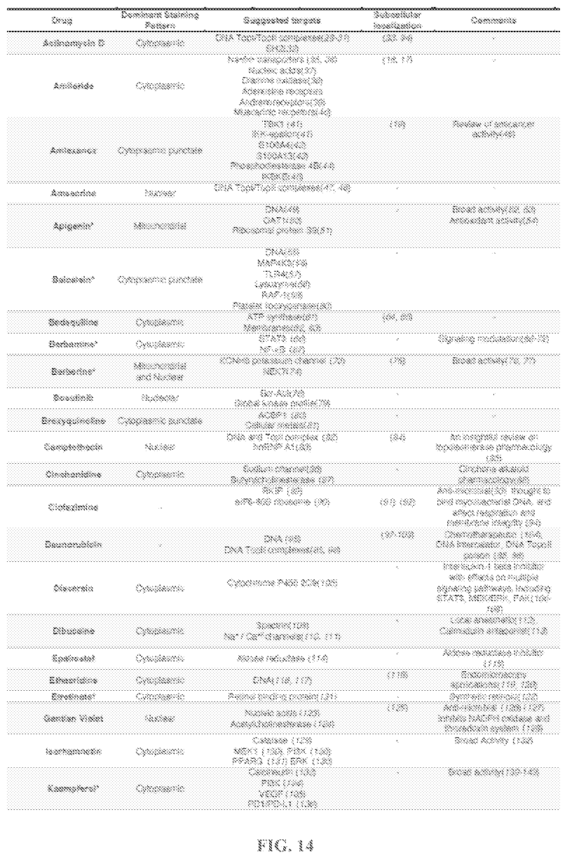

To investigate the selective partitioning behavior of a larger set of molecules, we compared the partition ratios of probes that enriched in each condensate with those obtained in other condensates. We found that probes that partitioned above the 90th percentile partitioned into the other condensates at lower percentiles (FIGS. 2F-H). Furthermore, the partition ratios of high partitioning probes in these condensates were generally greater than the partition ratios in the other condensates, with some exceptions (FIGS. 10A-C). Probes in the lowest percentiles of partition ratios in each condensate tended to show higher partition ratios in the other two condensates (FIGS. 10D-I). This selective concentration of a distinct subset of probes in these condensates is consistent with the notion that the three condensates harbor distinct chemical environments that optimally solvate certain small molecules due to their specific chemical features.

### Shared Probe Features Suggest a Chemical Grammar in Biomolecular Condensates

We reasoned that there must be physicochemical rules that govern small molecule partitioning into the chemical environment of each condensate (FIG. 3A). Because solvents tend to best solvate molecules with chemical properties like those of the solvent, we expected small molecules with similar chemical features to partition similarly into any one condensate. As a test of this expectation, we first explored the chemical similarity of probes across the small molecule library by representing each probe as a bit vector with components indicating the presence or absence of a chemical feature, as represented by a Morgan Fingerprint (16), and then computed the Tanimoto similarity metric (92) between each pair of probes (FIG. 3B). We then ordered all probes by their partition ratio K for each condensate and generated a pairwise similarity matrix for each condensate (FIG. 3B). These matrices show comparisons of both the chemical similarity shared between two molecules and their respective partition ratios and were ordered from high-to-low K (from top to bottom). Because each similarity matrix is ordered by probe partition ratio in any one condensate, we can visualize if chemical similarity is associated with probe partition ratio (FIGS. 3C-D). Inspection of the Tanimoto similarity matrixes (FIG. 3C, FIGS. 11A-C) and quantification of mean Tanimoto similarity of each probe (FIG. 3C) in each condensate confirmed that pairs of probes that shared high partitioning behaviors (e.g., data points in the top left corner of each matrix) tended to be more chemically similar to one another than probe pairs with very different partition ratios (e.g., data points in the bottom left corner of each similarity matrix). These results are consistent with the notion that there must be rules for the chemical features of small molecules that engender an apparent attraction to the chemical environment of a specific condensate.

Because the highest partitioning probes for any one condensate showed some degree of chemical similarity (FIGS. 3C-D), and the partitioning behavior of many small molecules is condensate-specific (FIGS. 2A-H), we expected that high partitioning probes of any one condensate would be more similar to one another than to the high partitioning probes of another condensate. The results of such comparisons for the MED1, NPM1 and HP1α condensates confirmed this expectation (FIGS. 3E-F and 11D-G). These results are consistent with the idea that different condensates harbor different chemical solvation environments and that these cause small molecules with chemically similar features to selectively concentrate within these condensates.

### Deep Learning Discovers Compounds with Selective Partitioning Behaviors

The evidence that protein condensates possess distinct chemical solvation environments for small molecules. together with evidence that there are chemical similarities to the molecules that concentrate optimally in these condensates, suggests that a deep learning approach might be able to predict whether small molecules will concentrate in any one condensate. In this disclosure, a deep learning approach is disclosed that can predict whether small molecules concentrate in any one condensate. Deep learning-based small molecule property prediction employs chemical structures and phenotypic data and has proven successful in identifying small molecules with desirable properties (17). Training a deep learning message passing neural network (MPNN) on a small molecule's structure and its measured partition ratio for each of the different condensates could optimize the discovery of compounds with chemical properties that cause their partitioning within a condensate.

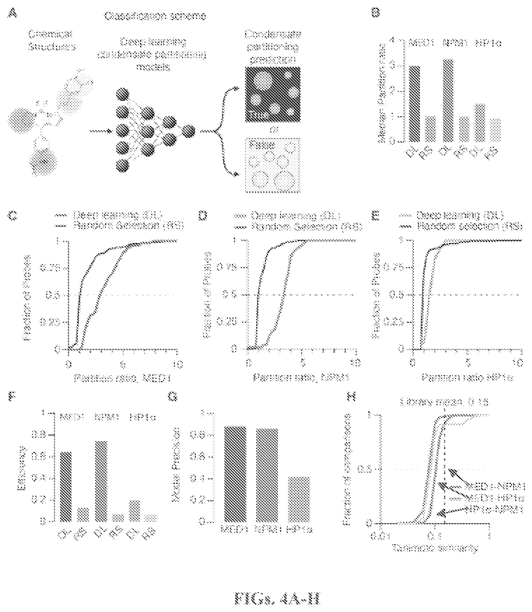

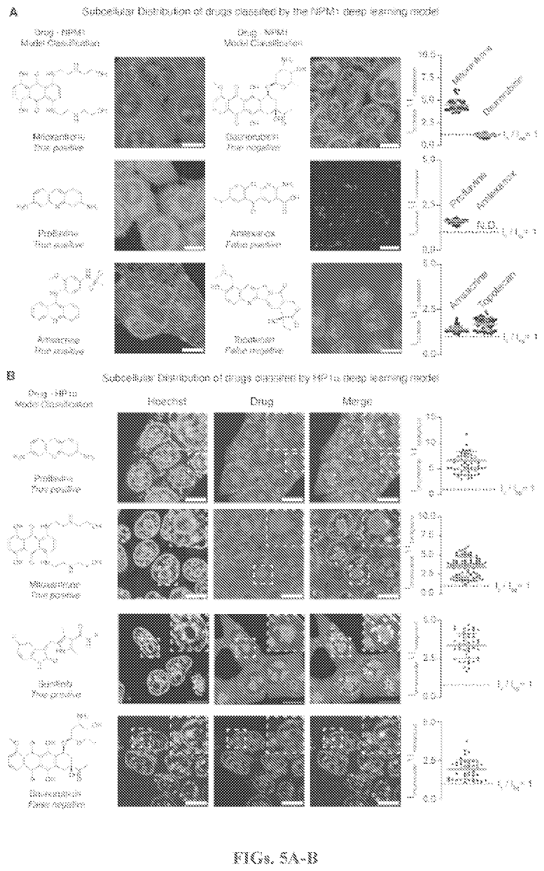

Deep learning MPNNs and random forests were trained and validated on the probe structures and binarized partitioning data for each of the MED1, NPM1 and HP1α protein condensates (FIG. 4A). Models were subsequently used to predict compounds with selective partitioning behavior from a set of probes withheld from the data for model development. We then used the in vitro condensate imaging assay to measure the partition ratios of predicted high partitioning probes in the withheld set of molecules and, as a control, 240 probes randomly selected from this set (FIG. 4B). A plot of the cumulative distribution functions of the experimentally determined probe partition ratios showed a shift in the median partition ratio toward higher partitioning for deep learning-selected probes as compared to those probes selected randomly (FIGS. 4C-E). These results indicate that deep learning can predict whether small molecules will concentrate in these condensates.

Deep learning was more efficient than random selection by 4-fold (MED1), 10-fold (NPM1), and 3-fold (HP1α) at identifying probes with partition ratios greater than their model training thresholds, K_{MED1} and K_{NPM1}>2.7, K_{HP1α}>2.0 (FIG. 4F). The greater efficiency of our MED1 and NPM1 models was concomitant with a greater proportion of true positive predictions, or a greater precision (FIG. 4G). When comparing probes identified by deep learning for one condensate versus another, at least 90% of the probes had a pairwise Tanimoto similarity less than the library mean (FIG. 4H). These results demonstrate that the chemical features of small molecules that lead to partitioning into various condensates can be identified with deep learning models and suggest that the rules for chemical features of small molecules that engender attraction to the chemical environment of a specific condensate can be learned and embedded in parameterized representations by neural networks.

### Condensate Chemical Environments Determine the Distribution of Small Molecules in Cells

We have observed that therapeutic small molecules can concentrate in subcellular compartments, including well-established biomolecular condensates (FIG. 1). Simple in vitro condensates formed by key scaffold proteins harbored distinct chemical environments that selectively partition small molecules (FIGS. 2A-H and 3A-F) and deep learning could predict molecules that selectively partition into condensates (FIGS. 4A-H). We wondered whether the chemical environments of these simple condensates might be sufficiently retained in the more complex condensates in living cells such that predictions based on partitioning in vitro might have predictive value in vivo. The billions of molecules in cells provide many opportunities for competitive interactions with small molecules that would be expected to limit our ability to translate the predictions of partitioning formed from in vitro experiments. However, previous studies with simple condensates suggest that such model systems can be predictive of the partitioning behaviors of small and large molecules in the more complex condensates that occur in cells (11, 18-23).

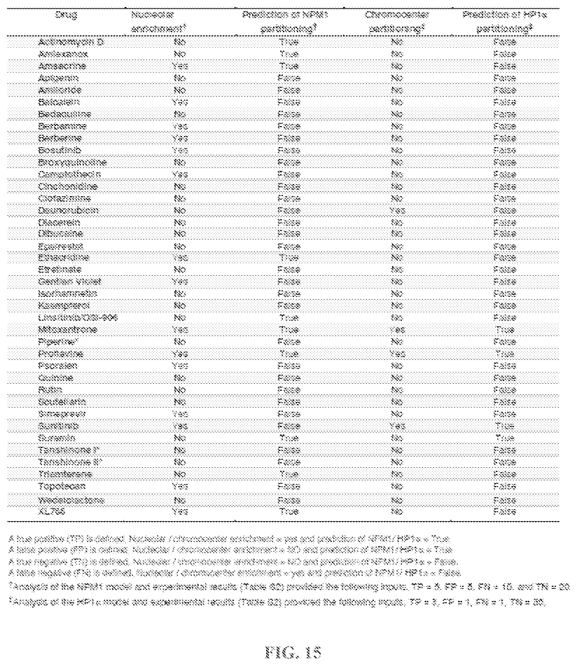

NPM1 is a scaffold protein for the nucleolus, so we investigated the extent to which the deep learning classifier, trained on probe partitioning data from NPM1 in vitro condensates, would correctly predict FDA approved drugs and natural products that concentrate in nucleolar condensates, which are straightforward to visualize due to their location, size and morphology. Of the 10 drugs predicted to concentrate in nucleoli, 5 were observed to do so (FIG. 5A, FIG. 15), and of the 30 drugs predicted not to concentrate in nucleoli, 11 appeared to concentrate in these bodies. This reveals that the model's predictions for the nucleolar condensate in living cells are less successful than those for the simple in vitro condensate, yet the model was 63% accurate and 2-fold better at correctly predicting high partitioning small molecules than an averaged random selection process, as determined by their diagnostic odds ratio.

HP1α is a scaffold protein for heterochromatin condensates that can be observed as chromocenters in murine embryonic stem cells (mESCs) (24), so we used the deep learning classifier, trained on probe partitioning data from HP1α in vitro condensates (FIGS. 2A-H), to predict small molecules that concentrate in chromocenters. Three of the four drugs predicted to have this behavior were found to concentrate in these chromocenters (FIG. 5B, FIG. 15). Among 36 other drugs tested that were not predicted to concentrate in chromocenters, only daunorubicin was found in chromocenters. These results show that the HP1α deep learning classifier was 95% accurate and ˜100-fold better at correctly predicting high partitioning small molecules than an averaged random selection process, as determined by their diagnostic odds ratio. The ability of the deep learning approach to predict with some accuracy that some drugs will selectively concentrate in the more complex environment of the relevant condensates in cells suggests that the chemical environment observed in simple in vitro condensates is retained to a degree in the more complex condensates in living cells.

### DISCUSSION

Data disclosed herein shows that small molecule therapeutics tend to concentrate in distinct intracellular compartments and that biomolecular condensates contain distinct chemical solvation environments that can selectively concentrate small molecules. The chemical features of small molecules that engender attraction to the chemical environment of a specific condensate can be predicted by using deep learning with small molecule probes. These results have important implications for our understanding of molecular interactions within cells and for improving the pharmacological activity of therapeutics.

Much of our understanding of biological regulatory mechanisms has been established by identifying the collection of protein and other biomolecules that bind to one another with high affinity (e.g., K_{d} between 100 pM-1 μM) relative to their interactions with other biomolecules, thus producing complexes of specific molecules with a certain stoichiometry and stability. By contrast, dynamic, multivalent low affinity interactions generated by the ensemble of diverse biomolecules in condensates can produce distinct internal chemistries. The different chemical environments of biomolecular condensates may thus confer additional specificity on biological regulatory processes beyond those obtained through canonical high-affinity interactions.

The evidence that condensates harbor distinct chemical environments implies that the selective incorporation of specific biomolecules into particular condensates is likely to be governed both by the solvation environment produced by the ensemble of components in the condensate and by high-affinity interactions with other biomolecules. Similarly, these results imply that two independent mechanisms can contribute to selective concentration of drugs in specific intracellular compartments: interactions with the chemical environment of diverse condensates and high-affinity interactions with specific portions of target proteins.

The chemical solvation properties of simple in vitro protein condensates, inferred by deep learning, could be used to predict with some accuracy the tendency of small molecule drugs to concentrate in the more complex condensate where that protein serves as a scaffold in living cells. It is possible that the scaffold proteins selected for study tend to dominate the chemical environment in the more complex cellular condensate and/or tend to interact with other proteins or nucleic acids that favor similar chemical environments.

Machine learning was able to efficiently predict molecules that partition into in vitro condensates and when applied to FDA drugs and natural products it could predict the partitioning behavior of these molecules into the nucleolus of live cells, albeit with limited performance. But why would partitioning into in vitro condensates be predictive of partitioning in live cells? Several possible models could explain these results. 1) Similar concentrations of the condensate scaffolding protein occur within condensates in vitro and in vivo, so that the chemical environments which concentrate a molecule are present in similar amounts in both cases. 2) The physicochemical properties of condensates in vitro and in vivo cause the intrinsically disordered regions of proteins to populate longer-lived transient structures inside of condensates than those occupied outside of condensates. The longer lifetime of these states inside of a condensate leads to favorable interactions with small molecules, which concentrates them within the condensate. 3) The insides of condensates create a unique solvation environment distinct from the environment composing the external milieu. In vitro and in vivo, this solvation environment favorably interacts with small molecules and other client proteins, and because chemically similar molecules solvate each other most favorably, some chemical features are more favorable than others for molecules to concentrate within a condensate. This is a restatement of like-dissolves-like, for the complex internal chemical solvation environments of a condensate as it applies to molecules which concentrate within that condensate. In each of the cases above, the mechanism by which small molecules concentrate within condensates leads to the selectivity of condensate for small molecules.

The mutual concentration of small molecule therapeutics and their target proteins in a specific condensate would be expected to create optimal therapeutic efficacy. However, we observed multiple instances where a therapeutic concentrated in a subcellular compartment unrelated to the location of the target protein of that drug (FIG. 1, FIG. 14, FIGS. 5A-B). For example, much of the fluorescent signal of the tyrosine kinase inhibitor sunitinib occurred in the mitochondria and the nucleolus, but the target receptor tyrosine kinase is thought to reside in the plasma membrane. Drug uptake into compartments that do not contain the target may lead to off-target interactions and, in some cases, toxicity. We propose that through improved understanding condensate chemical grammar, the chemical features of a drug might be optimized to enhance its concentration in target-containing condensates while reducing its concentration in off-target compartments, resulting in small molecule therapeutics with improved pharmacodynamic profiles.

### Materials and Methods

### Tumor Cell Tissue Culture

Human colorectal cancer cells (HCT-116 American Tissue Culture Catalog CCI-247™) were cultured in sterile 10 or 15 cm plates with 15 or 35 mL of DMEM (Gibco, 11965084) media supplemented with 10% Fetal bovine serum (FBS) (Sigma F2442) and 100 units/mL penicillin (Life Technologies, 15140122), and 100 μg/mL streptomycin (Life Technologies, 15140122). Cells were cultured at 37° C. and 5% v/v CO₂ in a humidified cell culture incubator and passaged at 75% confluency. Cells were counted to determine seeding density using a Countess™ II automated cell counter, employing trypan blue and disposable countess chamber slides according to manufacturer recommendations. Cells were tested regularly for mycoplasma using the MycoAlert Mycoplasma Detection Kit (Lonza LT07-218) and found to yield negative results. HCT-116 cells expressing MED1-, NPM1-, and HP1α-GFP from the endogenous gene locus were previously reported (11).

### Mouse Embryonic Stem Cell Tissue Culture

V6.5 mouse embryonic stem cells (mESCs) were a kind gift from R. Jaenisch, and were authenticated by STR analysis compared to commercially acquired cells with the same name. Stem cells were cultured in 2i/LIF medium on tissue culture-treated plates coated with 0.2% gelatin (Sigma G1890) in a humidified incubator at 37° C. and 5% CO2. Cells were passaged every 1-2 days by dissociation using TrypLE Express (Gibco 12604) and the dissociation reaction was quenched using serum/LIF medium. Cells were tested regularly for mycoplasma using the MycoAlert Mycoplasma Detection Kit (Lonza LT07-218) and found to yield negative results.

2i/LIF medium is defined as 3 μM CHIR99021 (Stemgent 04-0004), 1 μM PD0325901 (Stemgent 04-0006), and 1000 U-1 mL leukemia inhibitor factor (LIF, ESGRO ESG1107) in N2B27 medium. The composition of N2B27 medium is as follows: DMEM/F12 (Gibco 11320) supplemented with 0.5-fold N2 supplement (Gibco 17502), 0.5-fold B27 supplement (Gibco 17504), 2 mM L-glutamine (gibco 25030), 1-fold MEM non-essential amino acids (Gibco 11140), 100 U-1 mL penicillin-streptomycin (Gibco 15140), and 0.1 mM 2-mercaptoethanol (Sigma m7522).

Serum/LIF medium was prepared from KnockOut DMEM (Gibco 10829) supplemented with 15% fetal bovine serum (Sigma F4135), 2 mM L-glutamine (Gibco 25030), 1-fold MEM non-essential amino acids, 100 U-1 mL penicillin-streptomycin, 100 μM 2-mercaptoethanol (Sigma M7522) and 1000 U-1 mL LIF (ESGRO ESG1107).

### Instrumentation

Droplet images were recorded with an Andor Revolution spinning disk confocal microscope using a 1.4 NA 100× Plan Apo objective and a 150× zoom function in screening mode. The Andor revolution was outfit with an Andor iXion+EMCCD camera and excitation lasers at 50 mW 405, 50 mW 488, 50 mW 561 nm, 100 mW 640 nm. Emission intensity was collected with bandpass EM-CCD band pass filters 405 nm (447/60 nm), 488 (525/40 nm), 561 (617/73 nm), 640 (685/41 nm). Excitation intensity was maintained constant throughout all screening experiments.

Live cell confocal micrographs were recorded with a Zeiss LSM 980 Airyscan 2 Laser Scanning confocal operating in super resolution mode with a 1.4 NA 63× Plan Apo objective. Cells were maintained at 37° C. and 5% v/v CO₂ in a humidified chamber throughout the experiment with accompanying atmospheric controls. Images were recorded using 405 nm 25 mW, 488 nm 25 mW, 561 25 mW, or 639 nm 25 mW diode laser. Excitation intensity was adjusted according to analyte brightness.

Live cell Two-photon micrographs were recorded with a Zeiss LSM 710 Laser Scanning confocal operating in 2-photon mode with a 1.4 NA 63× Plan Apo Objective. Cells were maintained at 37° C. and 5% v/v CO₂ in a humidified chamber throughout the experiment with accompanying atmospheric controls. Images were recorded using Coherent Chameleon Ultra II femtosecond pulsed-IR laser, tuned to 750 nm. Excitation intensity was adjusted according to analyte brightness. Images were averaged twice.

### Live Cell Imaging

HCT-116 cells or endogenously tagged NPM1-GFP HCT-116 cells were seeded at 200,000 cells/mL on an imaging plate. Imaging plates used were sterile Cellvis 96-well glass (Cellvis, P96-1.5H-N) bottom plates with #1.5 high performance cover glass (0.17±0.005 mm), or sterile Cell vis 384-well (Cellvis, P384-1.5H-N) glass bottom plates with #1.5 high performance cover glass (0.17±0.005 mm).

Cells were plated 24 hours prior to the experiment. Prior to imaging, cells were washed once with fresh DMEM (Gibco, 11965084) supplemented with FBS/PS (Life Technologies, 15140122), 4.5 g/L glucose, 110 mg/mL sodium pyruvate, and 584.4 mg/mL L-glutamine. Then a premixed solution of analyte at a given concentration was prepared at a concentration of 5 to 100 μM in DMEM supplemented with FBS/PS and then incubated with cells. The analyte solution was allowed to incubate with the cells for 10 minutes at 37° C. and 5% v/v CO₂, prior to a final wash and application of fresh DMEM supplemented with FBS/PS followed by imaging. Cells were maintained at 37° C. with 5% v/v CO₂ in a humidified chamber over the course of the imaging experiment.

Mouse embryonic stem cells were imaged on sterile Cellvis 96-well glass (Cellvis, P96-1.5H-N) bottom plates with #1.5 high performance cover glass (0.17±0.005 mm), or sterile Cell vis 384-well (Cellvis, P384-1.5H-N) glass bottom plates with #1.5 high performance cover glass (0.17±0.005 mm). These plates were coated with poly-L-ornithine (Sigma P4957) for 30 minutes at 37° C. followed by a coating with 20 μg/mL laminin (Corning 354232) for 2 hours at 37° C. Cells were maintained at 37° C. with 5% v/v CO₂ in a humidified chamber over the course of the imaging experiment

### Small Molecule Fluorescent Probe Library

The small molecule fluorescent probe library consisted of a pool of 6000 fluorescent dyes. The library consisted of xanthene, xanthone, boron dipyrromethene (BODIPY), and cyanine dyes. Selection of probes for experiments was made by the fluorophore and microscope optical constraints. Fluorescent probes were maintained at a concentration of 10 mM in DMSO then diluted to 10 μM prior to use in in vitro screening assays.

### Recombinant Protein Expression and Purification

For protein expression plasmids were transformed into LOBSTR cells (a kind gift of Chessman Lab) and grown as follows. A fresh bacterial colony was inoculated into LB media containing kanamycin and chloramphenicol and grown overnight at 37° C. Cells were diluted 1:30 in 500 mL room temperature LB with freshly added kanamycin and chloramphenicol and grown 2.5 hours at 16° C. IPTG was added to 1 mM and growth continued for 20 hours. Cells were collected and stored frozen at −80° C.

Pellets from 500 mL cells were resuspended in 15 mL of Buffer A (50 mM Tris pH7.4, 500 mM NaCl), complete protease inhibitors (Roche, 11873580001) and sonicated (ten cycles of 15 seconds on, 60 sec off). The lysate was cleared by centrifugation at 12,000 g for 30 minutes at 4° C. and added to 1 mL of Ni-NTA agarose (Invitrogen, R901-15) pre-equilibrated with 10× volumes of buffer A. Tubes containing this agarose lysate slurry were rotated at 4° C. for 1.5 hours. The slurry was centrifuged at 3,000 rpm for 10 minutes. The resin was washed with 2×5 mL of Buffer A followed by 2×5 mL Buffer A containing 50 mM imidazole. The protein was eluted by rotating with 3× with 2 mL Buffer A containing 250 mM imidazole incubating rotating for 10 or more minutes each cycle at 4° C. Each eluate was run on a 12% Bis-Tris acrylamide gel. Fractions containing protein of the correct size were dialyzed against two changes of buffer containing 50 mM Tris 7.4, 500 mM NaCl, 10% glycerol and 1 mM DTT at 4° C. Any precipitate after dialysis was removed by centrifugation at 3,000 rpm for 10 minutes.

### In Vitro Droplet Assay

Purified recombinant MED1-BFP, HP1α-BFP, and NPM1-BFP fusion proteins were purified and concentrated to 50 μM as described above. Protein was added to a droplet formation buffer consisting of 50 mM Tris HCL, 1 mM DTT, 125 nM NaCl, 10% 8 kDa polyethylene glycol crowding agent at pH 7.5. A Tecan Evo 150 or a Beckman Echo 655 liquid handler was used to dispense 50 nL of fluorescent probe from a master plate containing fluorescent probes at 10 mM in DMSO, to a solution of 1 μL 50 μM protein and 9 μL buffer solution as described above. The plate was sealed with parafilm, protected from light and incubated at 37° C. overnight to equilibrate the sample. After equilibration, droplet images were recorded at room temperature using the plate screening mode with the Andor microscope as described above. In total, 11 image were recorded for each fluorescent probe at different locations within the image with 500 ms exposures and a normalized laser power.

### Droplet Image Analysis

Droplet image analysis was performed using an inhouse developed python script. Briefly, a binary mask was generated from the 405 nm or protein channel from signal that was of at least 25 pixels in size and with intensity values above the background of each image (droplets were detected from the 405 nm excitation channel). The intensity of the fluorescent probe was measured within and outside of the regions demarcated by this mask in the fluorescent probe channels (488, 561, 640 nm) and averaged. The concentration of a fluorescent probe was assumed to be proportional to the intensity of the fluorescent probe inside and outside of the binary mask, and the partition ratio, K, was computed as Intensity≈C, for C=Cᵢₙ or Cₒᵤₜ as defined by the binary mask. The partition ratio used here is the quotient of these values Cᵢₙ/Cₒᵤₜ=K. The total number of probes used in MED1, NPM1 and HP1α droplets were 1143, 1055, and 963 molecules, respectively. Measurements of protein condensed fraction were performed by computing the area in each in the 405 nm channel (protein droplet detection channel) with a fluorescent intensity above the background fluorescence intensity and comparing this value against the total area of each image.

### Chemoinformatics

Fluorescent probe chemical structures were generated as SMILES strings and sanitized. Pairwise Tanimoto similarity calculations were performed using Morgan Fingerprints with a radius of 2 in a 2048-bit depth as implemented in the program RDKit (v2021.03). (25)

### Machine Learning

Datasets quantifying the partitioning of small molecules in MED1, NPM1 and HP1α droplets were collected, the datasets consisting of 1143, 1055, and 963 molecules, respectively. To predict the partitioning ratio of molecules, a random forest classifier and a directed message-passing neural network (MPNN) were trained separately and their respective predictions (e.g., outputs) are aggregated. Given a molecule's SMILES string, the models aimed to predict if the molecule's partition ratio was above a preset threshold. A threshold can be selected (e.g., by a user, designer, etc.) for each condensate: 2.7 for MED1, 2.7 for NPM1, and 2.0 for HP1α to select compounds which partition into a condensate, or not.

The random forest classifiers were trained using the scikit-learn package (v0.24.2) in Python (v3.8.10), setting “n_estimators” to 200, “min_samples_leaf” to 2, and “n_jobs” to 4 (26) Each molecule was transformed into a 1024-dimensional vector using the Chem.RDKFingerprint method from the open-source package RDKit (v2021.03.2) (25). Each classifier was trained on 90% of the data. To train the MPNN models on the classification tasks, we used Chemprop (v1.3.1) (27). The models took as input both the SMILES string representation of each molecule as well as a 200-dimensional vector generated using Chemprop and setting “features_generator” to rdkit_2d_normalized. Molecules were assigned to either the training set (80%), validation set (10%), or test set (10%) using a scaffold split. All MPNNs were trained with a batch size of 50 for 50 epochs with an ensemble of 10 models per task.

Predictions for a held-out dataset of 1,498 fluorescent molecules were determined by majority voting. A molecule's partitioning ratio was predicted to be above a given threshold if both the random forest and MPNN models predicted a score greater than 0.5. For molecule partitioning rations that are predicted to be below the given threshold by at least one of the random forest and MPNN, the molecule's partitioning ratio will be predicted to be below the given threshold.

### Drug Nucleolar Enrichment

A drug was classified as enriched if a distinct nucleolar pattern could be observed in a cell and considered as unenriched if a nucleolar pattern could not be observed. Systems measured the intensity of signal from endogenously fluorescent drugs in regions discernable as the nucleolus to across 3 different images and between 5-15 cells to compute in the intensity of light in the nucleolus, Iₙ, and compared it to the intensity of the light in the nucleoplasm to describe a molecule as enriched if the mean nucleolus Iₙ/Iₙₚ>1.10. Enriched or unenriched populations of each molecule were then used in the statistical analyses of the model's performance (see FIG. 15 and statistical analysis).

### Drug Chromocenter Enrichment

Cells were treated with Hoechst 33342 at 0.1 μg/mL and 50 μM of an endogenously fluorescent small molecule in 2i/LIF media for 10 minutes at 37° C. and 5% CO2 in a L-ornithine and laminin treated glass bottom plate or dish. Cells were then taken out of the incubator, washed twice with fresh 2i/LIF media and fresh 2i/LIF media was placed on the cells. Images were then recorded as described above using a confocal or two-photon microscope and analyzed using Fiji. At least fifty chromocenters were analyzed across 5-10 images by selecting large punctate structures demarcated by Hoechst 33342 stain and the intensity of signal in these objects (I_{chromocenter}) was measured in the 405 nm and 488, 561, or 639 nm channels to assess the presence of Hoechst or the drug respectively. The background intensity (I_{background}) was determined by selecting 50 regions in different cells where the nucleus not marked by Hoechst stain, and the intensity of signal in these regions was measured using the 405 nm and 488, 561, or 639 nm channels to assess the presence of Hoechst or the drug respectively. Chromocenter partitioning was evaluated by taking the ratio of I_{chromocenter}/I_{background}, and a chromocenter was considered enriched in a drug if I_{chromocenter}/I_{background}>1.10. The enrichment of a molecule in each chromocenter was then used in the assessment of model performance (see FIG. 15 and statistical analysis).

### Statistical Analysis

All statistical tests were performed using GraphPad Prism (v. 9.2.0). Comparisons between partition ratio distributions (FIG. 2F-H, FIG. 10G-I) were made using Wilcoxon matched-pairs signaed rank test. Comparisons between distributions of partition ratio percentiles (FIGS. 10A-F) were analyzed using a Wilcoxon matched-pairs signed rank test. Differences in mean Tanimoto similarity distributions (FIGS. 3F and 3G) were evaluated using a paired t-test. To assess classifier performance on nucleolar enrichment (FIG. 4J), the metrics of accuracy (ACC), balanced accuracy (BA), F1-score (F1), informed-ness (J), and diagnostic odds ratio (DOR) were computed as follows:

$$ACC = \frac{TP + TN}{TP + TN + FP + FN} \\ BA = \frac{TPR + TNR}{2} \\ F 1 = \frac{2 * TP}{2 * TP + FP + FN} \\ J = TPR + TNR - 1 \\ DOR = \frac{TP * TN}{FP * FN} \\ where : \\ TPR = \frac{TP}{TP + FN} \\ TNR = \frac{TN}{TN + FP}$$

With TP=True positive, TN=True negative, FP=False positive, FN=False negative. The 95% confidence interval for DOR was computed assuming that the In (DOR) followed a normal distribution.

A true positive (TP) is defined, nucleolar/chromocenter enrichment=yes and prediction of NPM1/HP1α=true, a false positive (FP) is defined, nucleolar/chromocenter enrichment=no and prediction of NPM1/HP1α=true. And a true negative (TN) is defined, nucleolar/chromocenter enrichment=no and prediction of NPM1/HP1α=false. A false negative (FN) is defined, nucleolar/chromocenter enrichment=yes and prediction of NPM1/HP 1α=false.

Analysis of the NPM1 model and experimental results (FIG. 15) provided the following inputs, TP=5, FP=5, FN=10, and TN=20 and thus we found a TPR=0.33, TNR=0.80, an accuracy (ACC)=0.63, balanced accuracy (BA)=0.57, F1=0.40, an informed-ness (I)=0.13, DOR=2 (95% CI, 0.46-8.55). The NPM1 model was 10% more accurate than a random model as computed below, and had a 2-fold greater DOR.

Analysis of the HP1α model and experimental results (FIG. 15) provided the following inputs, TP=3, FP=1, FN=1, TN=35, and thus we found a TPR=0.75, TNR=0.97, an accuracy (ACC)=0.95, balanced accuracy (BA)=0.86, F1=0.75, an informed-ness (I)=0.72, DOR=105 (95% CI, 5-2,135). The HP1α model was 45% more accurate than random model as computed below, and had a 105-fold greater DOR.

The DOR of the NPM1 and HP1α models was compared to a ‘random model’ defined such that pool of compounds was a total of 40 split evenly across each different input, i.e., TP=TN=FP=FN=10, which provides a DOR=1 and an accuracy of 0.50

### REFERENCES

1. Y. Shin, C. P. Brangwynne, Liquid phase condensation in cell physiology and disease. Science 357, (2017).
2. E. M. Langdon, A. S. Gladfelter, A New Lens for RNA Localization: Liquid-Liquid Phase Separation. Annu. Rev. Micrbiol. 72, 255-271 (2018).
3. A. S. Lyon, W. B. Peeples, M. K. Rosen, A framework for understanding the functions of biomolecular condensates across scales. Nat. Rev. Mol. Cell Biol. 22, 215-235 (2021).
4. S. Alberti, A. A. Hyman, Biomolecular condensates at the nexus of cellular stress, protein aggregation disease and ageing. Nat. Rev. Mol. Cell Biol. 22, 196-213 (2021).
5. M. Feric, T. Misteli, Phase separation in genome organization across evolution. Trends Cell Biol. 31, 671-685 (2021).
6. J. D. Forman-Kay, J. A. Ditlev, M. L. Nosella, H. O. Lee, What are the distinguishing features and size requirements of biomolecular condensates and their implications for RNA-containing condensates? RNA 28, 36-47 (2022).
7. M. Du, J. Chen Zhijian, DNA-induced liquid phase condensation of cGAS activates innate immune signaling. Science 361, 704-709 (2018).
8. H. R. Kilgore, R. A. Young, Learning the chemical grammar of biomolecular condensates. Nat. Chem. Biol., 10.1038/s41589-41022-01046-y (2022).
9. B. G. O'Flynn, T. Mittag, The role of liquid-liquid phase separation in regulating enzyme activity. Curr. Opin. Cell Biol. 69, 70-79 (2021).
10. W. Peeples, M. K. Rosen, Mechanistic dissection of increased enzymatic rate in a phase-separated compartment. Nat. Chem. Biol. 17, 693-702 (2021).
11. I. A. Klein, et. al., Partitioning of cancer therapeutics in nuclear condensates. Science 368, 1386 (2020).
12. T. P. Howard, C. W. M. Roberts, Partitioning of Chemotherapeutics into Nuclear Condensates; Opening the Door to New Approaches for Drug Development. Mol. Cell 79, 544-545 (2020).
13. B. R. Sabari et al., Coactivator condensation at super-enhancers links phase separation and gene control. Science 361, eaar3958 (2018).
14. A. G. Larson et al., Liquid droplet formation by HP1α suggests a role for phase separation in heterochromatin. Nature 547, 236-240 (2017).
15. A. Patel et al., A Liquid-to-Solid Phase Transition of the ALS Protein FUS Accelerated by Disease Mutation. Cell 162, 1066-1077 (2015).
16. D. Rogers, M. Hahn, Extended-Connectivity Fingerprints. J. Chem Inf. Model. 50, 742-754 (2010).
17. W. P. Walters, R. Barzilay, Applications of Deep Learning in Molecule Generation and Molecular Property Prediction. Acc. Chem. Res. 54, 263-270 (2021).
18. E. Y. Guo, et. al., Pol II phosphorylation regulates a switch between transcriptional and splicing condensates. Nature 572, 543-548 (2019).
19. A. V. Zamudio et al., Mediator Condensates Localize Signaling Factors to Key Cell Identity Genes. Mol. Cell 76, 753-766 (2019).
20. J. Wang, et. al., A molecular grammar governing the driving forces for phase separation of prion-like RNA binding proteins Cell 174, 688-699.e616 (2018).
21. L. B. Case, X. Zhang, J. A. Ditlev, M. K. Rosen, Stoichiometry controls activity of phase-separated clusters of actin signaling proteins. Science 363, 1093-1097 (2019).
22. H. Yu et al., HSP70 chaperones RNA-free TDP-43 into anisotropic intranuclear liquid spherical shells. Science 371, eabb4309 (2021).
23. B. Chandra et al., Phase Separation Mediates NUP98 Fusion Oncoprotein Leukemic Transformation. Cancer Discov. 12, 1152-1169 (2022).
24. A. R. Strom et al., Phase separation drives heterochromatin domain formation. Nature 547, 241-245 (2017).
25. G. Landrum. (2010), vol. RDKit: Open-Source chemoinformatics. Q2 2021.
26. F. Pedregosa et al., Scikit-learn: Machine Learning in Python. J. Mach. Learn. Res. 12, 2825-2830 (2011).
27. K. Yang et al., Analyzing Learned Molecular Representations for Property Prediction. J. Chem. Inf. Mod. 59, 3370-3388 (2019).
28. E. C. Cesconetto et al., DNA interaction with Actinomycin D: mechanical measurements reveal the details of the binding data. Phys. Chem. Chem. Phys. 15, 11070-11077 (2013).
29. S. L. Cravens, A. C. Navapanich, B. H. Geierstanger, D. C. Tahmassebi, T. J. Dwyer, NMR solution structure of a DNA-actinomycin D complex containing a non-hydrogen-bonding pair in the binding site. J. Am. Chem. Soc. 132, 17588-17598 (2010).
30. F. L. Yu, W. Bender, Actinomycin D binding in vitro: active chromatin preferred. Biochem. Int. 20, 807-815 (1990).
31. S. Toku, Y. Nabeshima, K. Ogata, Binding of actinomycin D to mRNA in vivo and in vitro. J. Biochem. 93, 361-366 (1983).
32. H. K. Kim et al., Actinomycin D as a novel SH2 domain ligand inhibits Shc/Grb2 interaction in B104-1-1 (neu*-transformed NIH3T3) and SAA (hEGFR-overexpressed NIH3T3) cells. FEBS Lett. 453, 174-178 (1999).
33. D. Sonneborn, H. Rothstein, Studies on the uptake and intracellular localization of 3H actinomycin D in lens epithelial cells. Biosystems 1, 186-188 (1967).
34. M. J. C. Hendrix, H. N. Wagner, S. P. Thomson, Immunohistochemical localization of actinomycin D in human melanoma tumor cells. Anticancer Res. 4, 97-102 (1984).
35. S. Chaudhary, P. Kumar, M. Kaushik, Exploring the interaction of guanidine ligands Amiloride, Rimeporide and Cariporide with DNA for understanding their role as inhibitors of Na (+)/H (+) exchangers (NHEs): A spectroscopic and molecular docking investigation. Int. J. Biol. Macromol. 213, 834-844 (2022).
36. O. Kelly et al., Characterization of an amiloride binding region in the alpha-subunit of ENaC. Am. J. Physiol. Renal Physiol. 285, F1279-1290 (2003).
37. Y. Sato, T. Ichihashi, S. Nishizawa, N. Teramae, Strong and selective binding of amiloride to an abasic site in RNA duplexes: thermodynamic characterization and microRNA detection. Angew. Chem. Int. Ed. Engl. 51, 6369-6372 (2012).
38. W. F. Novotny, O. Chassande, M. Baker, M. Lazdunski, P. Barbry, Diamine oxidase is the amiloride-binding protein and is inhibited by amiloride analogues. J. Biol. Chem. 269, 9921-9925 (1994).
39. S. M. Periyasamy, Interaction of amiloride with alpha-adrenoreceptors: evidence from radioligand binding studies. Can. J. Physiol. Pharmacol. 66, 596-600 (1988).
40. J. P. Dehaye, V. Verhasselt, Interaction of amiloride with rat parotid muscarinic and alpha-adrenergic receptors. Gen. Pharmacol. 26, 155-159 (1995).
41. S. M. Reilly et al., An inhibitor of the protein kinases TBK1 and IKK-ε improves obesity-related metabolic dysfunctions in mice. Nat. Med. 19, 313-321 (2013).
42. C. C. Cho, R. H. Chou, C. Yu, Amlexanox Blocks the Interaction between S100A4 and Epidermal Growth Factor and Inhibits Cell Proliferation. PLOS One 11, e0161663 (2016).
43. S. G. Rani, S. K. Mohan, C. Yu, Molecular level interactions of S100A13 with amlexanox: inhibitor for formation of the multiprotein complex in the nonclassical pathway of acidic fibroblast growth factor. Biochemistry 49, 2585-2592 (2010).
44. Y. Han et al., Amlexanox exerts anti-inflammatory actions by targeting phosphodiesterase 4B in lipopolysaccharide-activated macrophages. Biochim. Biophys. Acta Mol. Cell Res. 1867, 118766-118766 (2020).
45. J. Xiong et al., Amlexanox Enhances Temozolomide-Induced Antitumor Effects in Human Glioblastoma Cells by Inhibiting IKBKE and the Akt-mTOR Signaling Pathway. ACS Omega 6, 4289-4299 (2021).
46. C. Bailly, The potential value of amlexanox in the treatment of cancer: Molecular targets and therapeutic perspectives. Biochem. Pharmacol. 197, 114895 (2022).
47. D. K. Jangir, S. Kundu, R. Mehrotra, Role of minor groove width and hydration pattern on amsacrine interaction with DNA. PLOS One 8, e69933 (2013).
48. A. C. Ketron, W. A. Denny, D. E. Graves, N. Osheroff, Amsacrine as a topoisomerase II poison: importance of drug-DNA interactions. Biochemistry 51, 1730-1739 (2012).
49. S. Waihenya et al., Mechanism of Interactions of dsDNA Binding with Apigenin and Its Sulfamate Derivatives Using Multispectroscopic, Voltammetric, and Molecular Docking Studies. ACS Omega 6, 5124-5137 (2021).
50. T. Wu et al., Apigenin, a novel candidate involving herb-drug interaction (HDI), interacts with organic anion transporter 1 (OAT1). Pharmacol. Rep. 69, 1254-1262 (2017).
51. Y. Iizumi et al., The flavonoid apigenin downregulates CDK1 by directly targeting ribosomal protein S9. PLOS One 8, e73219 (2013).
52. X. Zhou, F. Wang, R. Zhou, X. Song, M. Xie, Apigenin: A current review on its beneficial biological activities. J. Food Biochem. 41, e12376 (2017).
53. R. Avallone et al., Pharmacological profile of apigenin, a flavonoid isolated from Matricaria chamomilla. Biochem. Pharmacol. 59, 1387-1394 (2000).
54. K. Horváthová, D. Novotný L Fau-Tóthová, A. Tóthová D Fau-Vachálková, A. Vachálková, Determination of free radical scavenging activity of quercetin, rutin, luteolin and apigenin in H2O2-treated human ML cells K562. Neoplasma 51, 395-399 (2004).
55. R. Wang, J. Li, D. B. Niu, F. Y. Xu, X. A. Zeng, Protective effect of baicalein on DNA oxidative damage and its binding mechanism with DNA: An in vitro and molecular docking study. Spectrochim. Acta A Mol. Biomol. Spectrosc. 253, 119605 (2021).
56. J. Li et al., Baicalein suppresses growth of non-small cell lung carcinoma by targeting MAP4K3. Biomed. Pharmacother. 133, 110965 (2021).
57. M. Chen et al., Baicalein is a novel TLR4-targeting therapeutics agent that inhibits TLR4/HIF-1α/VEGF signaling pathway in colorectal cancer. Clin. Transl. Med. 11, e564 (2021).
58. D. Li, T. Zhang, C. Xu, B. Ji, Effect of pH on the interaction of baicalein with lysozyme by spectroscopic approaches. J. Photochem. Photobiol. B 104, 414-424 (2011).
59. N. Nakahata, C. Tsuchiya, K. Nakatani, Y. Ohizumi, S. Ohkubo, Baicalein inhibits Raf-1-mediated phosphorylation of MEK-1 in C6 rat glioma cells. Eur. J. Pharmacol. 461, 1-7 (2003).
60. K. Sekiya, H. Okuda, Selective inhibition of platelet lipoxygenase by baicalein. Biochem. Biophys. Res. Commun. 105, 1090-1095 (1982).
61. M. Fiorillo, C. Scatena, A. G. Naccarato, F. Sotgia, M. P. Lisanti, Bedaquiline, an FDA-approved drug, inhibits mitochondrial ATP production and metastasis in vivo, by targeting the gamma subunit (ATP5F1C) of the ATP synthase. Cell Death Differ. 28, 2797-2817 (2021).
62. K. N. Belosludtsev et al., Interaction of the anti-tuberculous drug bedaquiline with artificial membranes and rat erythrocytes. Chem. Biol. Interact. 299, 8-14 (2019).
63. M. C. Cholo, M. T. Mothiba, B. Fourie, R. Anderson, Mechanisms of action and therapeutic efficacies of the lipophilic antimycobacterial agents clofazimine and bedaquiline. J. Antimicrob. Chemother. 72, 338-353 (2017).
64. A. Fearns, D. J. Greenwood, A. Rodgers, H. Jiang, M. G. Gutierrez, Correlative light electron ion microscopy reveals in vivo localisation of bedaquiline in Mycobacterium tuberculosis-infected lungs. PLOS Biol. 18, e3000879-e3000879 (2020).
65. D. J. Greenwood et al., Subcellular antibiotic visualization reveals a dynamic drug reservoir in infected macrophages. Science 364, 1279-1282 (2019).
66. W. Zhao, B. Bai, Z. Hong, X. Zhang, B. Zhou, Berbamine (BBM), a Natural STAT3 Inhibitor, Synergistically Enhances the Antigrowth and Proapoptotic Effects of Sorafenib on Hepatocellular Carcinoma Cells. ACS Omega 5, 24838-24847 (2020).
67. Y. Liang, R.-z. Xu, L. Zhang, X.-y. Zhao, Berbamine, a novel nuclear factor KB inhibitor, inhibits growth and induces apoptosis in human myeloma cells. Acta Pharmacol. Sin. 30, 1659-1665 (2009).
68. X. Y. Zhao, Z. W. He, D. Wu, R. Z. Xu, Berbamine selectively induces apoptosis of human acute promyelocytic leukemia cells via survivin-mediated pathway. Chin. Med. J. 120, 802-806 (2007).
69. Y. L. Wei, Y. Liang, L. Xu, X. Y. Zhao, The antiproliferation effect of berbamine on k562 resistant cells by inhibiting NF-kappaB pathway. Anat. Rec. 292, 945-950 (2009).
70. X. J. Jia et al., Berbamine Exerts Anti-Inflammatory Effects via Inhibition of NF-κB and MAPK Signaling Pathways. Cell Physiol. Biochem. 41, 2307-2318 (2017).
71. A. Sharma, S. K. Anand, N. Singh, U. N. Dwivedi, P. Kakkar, Berbamine induced AMPK activation regulates mTOR/SREBP-1c axis and Nrf2/ARE pathway to allay lipid accumulation and oxidative stress in steatotic HepG2 cells. Eur. J. Pharmacol. 882, 173244 (2020).
72. L. Liu, Z. Xu, B. Yu, L. Tao, Y. Cao, Berbamine Inhibits Cell Proliferation and Migration and Induces Cell Death of Lung Cancer Cells via Regulating c-Maf, PI3K/Akt, and MDM2-P53 Pathways. Evid. Based Complement. Alternat. Med. 2021, U.S. Pat. No. 5,517,143 (2021).
73. M. M. Zhao et al., Berberine is an insulin secretagogue targeting the KCNH6 potassium channel. Nat. Commun. 12, 5616 (2021).
74. Q. Zeng et al., Berberine Directly Targets the NEK7 Protein to Block the NEK7-NLRP3 Interaction and Exert Anti-inflammatory Activity. J. Med. Chem. 64, 768-781 (2021).
75. M. Jin et al., Synthesis of a novel fluorescent berberine derivative convenient for its subcellular localization study. Bioorg. Chem. 101, 104021 (2020).
76. X. Lin, N. Zhang, Berberine: Pathways to protect neurons. Phytother. Res. 32, 1501-1510 (2018).
77. M. Chu et al., Polypharmacology of Berberine Based on Multi-Target Binding Motifs. Front. Pharmacol. 9, 801 (2018).
78. N. M. Levinson, S. G. Boxer, Structural and spectroscopic analysis of the kinase inhibitor bosutinib and an isomer of bosutinib binding to the Abl tyrosine kinase domain. PLOS One 7, e29828 (2012).
79. L. L. Remsing Rix et al., Global target profile of the kinase inhibitor bosutinib in primary chronic myeloid leukemia cells. Leukemia 23, 477-485 (2009).
80. J. M. Fritzler, G. Zhu, Novel anti-Cryptosporidium activity of known drugs identified by high-throughput screening against parasite fatty acyl-CoA binding protein (ACBP). J. Antimicrob. Chemother. 67, 609-617 (2012).
81. S. Y. Kim et al., Effects of Clioquinol Analogues on the Hypoxia-Inducible Factor Pathway and Intracelullar Mobilization of Metal Ions. Biol. Pharma. Bull. 35, 2160-2169 (2012).
82. F. Li, T. Jiang, Q. Li, X. Ling, Camptothecin (CPT) and its derivatives are known to target topoisomerase I (Top1) as their mechanism of action: did we miss something in CPT analogue molecular targets for treating human disease such as cancer? Am. J. Cancer Res. 7, 2350-2394 (2017).
83. D. Manita et al., Camptothecin (CPT) directly binds to human heterogeneous nuclear ribonucleoprotein A1 (hnRNP A1) and inhibits the hnRNP A1/topoisomerase I interaction. Bioorg. Med. Chem. 19, 7690-7697 (2011).
84. A. C. Croce et al., Subcellular localization of the camptothecin analogues, topotecan and gimatecan. Biochem. Pharmacol. 67, 1035-1045 (2004).
85. Y. Pommier, E. Leo, H. Zhang, C. Marchand, DNA Topoisomerases and Their Poisoning by Anticancer and Antibacterial Drugs. Chem. Biol. 17, 421-433 (2010).
86. R. J. Hill, H. J. Duff, R. S. Sheldon, Determinants of stereospecific binding of type I antiarrhythmic drugs to cardiac sodium channels. Mol. Pharmacol. 34, 659-663 (1988).
87. S. A. Nawaz, M. Ayaz, W. Brandt, L. A. Wessjohann, B. Westermann, Cation-π and π-π stacking interactions allow selective inhibition of butyrylcholinesterase by modified quinine and cinchonidine alkaloids. Biochem. Biophys. Res. Commun. 404, 935-940 (2011).
88. P. J. Boratyński, M. Zielińska-Błajet, J. Skarżewski, Cinchona Alkaloids-Derivatives and Applications. Alkaloids Chem. Biol. 82, 29-145 (2019).
89. C. Guo et al., Anti-leprosy drug Clofazimine binds to human Raf1 kinase inhibitory protein and enhances ERK phosphorylation. Acta Biochim. Biophys. Sin. 50, 1062-1067 (2018).
90. E. Pesce et al., Discovery and Preliminary Characterization of Translational Modulators that Impair the Binding of eIF6 to 60S Ribosomal Subunits. Cells 9, (2020).
91. D. Vandeputte, W. Jacob, R. Van Grieken, J. Boddingius, Study of intracellular deposition of the anti-leprosy drug Clofazimine in mouse spleen using laser microprobe mass analysis. Biol. Mass Spec. 22, 221-225 (1993).
92. J. Baik, G. R. Rosania, Macrophages Sequester Clofazimine in an Intracellular Liquid Crystal-Like Supramolecular Organization. PLOS One 7, e47494-e47494 (2012).
93. N. Riccardi et al., Clofazimine: an old drug for never-ending diseases. Future Microbiol. 15, 557-566 (2020).
94. M. C. Cholo, H. C. Steel, P. B. Fourie, W. A. Germishuizen, R. Anderson, Clofazimine: current status and future prospects. J. Antimicrob. Chemother. 67, 290-298 (2012).
95. T. Fukushima et al., Superior cytotoxic potency of mitoxantrone in interaction with DNA: comparison with that of daunorubicin. Oncol. Res. 8, 95-100 (1996).
96. H. M. Al-Aamri, H. R. Irving, T. Meehan-Andrews, C. Bradley, Determination of the DNA repair pathways utilised by acute lymphoblastic leukaemia cells following daunorubicin treatment. BMC Res. Notes 12, 625 (2019).
97. P. Mlejnek, J. Havlasek, N. Pastvova, P. Dolezel, Can image analysis provide evidence that lysosomal sequestration mediates daunorubicin resistance? Chem. Biol. Interact. 327, 109138 (2020).
98. J. Y. Yang et al., Subcellular daunorubicin distribution and its relation to multidrug resistance phenotype in drug-resistant cell line SMMC-7721/R. World J. Gastroenterol. 8, 644-649 (2002).
99. D. Lautier et al., Altered intracellular distribution of daunorubicin in immature acute myeloid leukemia cells. Int. J. Cancer. 71, 292-299 (1997).
100. A. Seidel et al., Intracellular localization, vesicular accumulation and kinetics of daunorubicin in sensitive and multidrug-resistant gastric carcinoma EPG85-257 cells. Virchows Arch. 426, 249-256 (1995).
101. J. E. Gervasoni, Jr. et al., Subcellular distribution of daunorubicin in P-glycoproteinpositive and -negative drug-resistant cell lines using laser-assisted confocal microscopy. Cancer Res. 51, 4955-4963 (1991).
102. A. A. Hindenburg et al., Intracellular distribution and pharmacokinetics of daunorubicin in anthracycline-sensitive and -resistant HL-60 cells. Cancer Res. 49, 4607-4614 (1989).
103. M. J. Egorin, R. C. Hildebrand, E. F. Cimino, N. R. Bachur, Cytofluorescence localization of adriamycin and daunorubicin. Cancer Res. 34, 2243-2245 (1974).
104. T. Murphy, K. W. L. Yee, Cytarabine and daunorubicin for the treatment of acute myeloid leukemia. Expert Opin. Pharmacother. 18, 1765-1780 (2017).
105. B. H. Tan et al., Cytochrome P450 2C9-natural antiarthritic interactions: Evaluation of inhibition magnitude and prediction from in vitro data. Biopharm. Drug Dispos. 39, 205-217 (2018).
106. K. Pavelka et al., Diacerein: Benefits, Risks and Place in the Management of Osteoarthritis. An Opinion-Based Report from the ESCEO. Drugs Aging 33, 75-85 (2016).
107. F. Domagala, G. Martin, P. Bogdanowicz, H. Ficheux, J. P. Pujol, Inhibition of interleukin-1beta-induced activation of MEK/ERK pathway and DNA binding of NFkappaB and AP-1: potential mechanism for Diacerein effects in osteoarthritis. Biorheology 43, 577-587 (2006).
108. M. Almezgagi et al., Diacerein: Recent insight into pharmacological activities and molecular pathways. Biomed. Pharmacother. 131, 110594 (2020).
109. M. Mondal, A. Chakrabarti, The tertiary amine local anesthetic dibucaine binds to the membrane skeletal protein spectrin. FEBS Lett. 532, 396-400 (2002).
110. M. Oka, Y. Itoh, T. Fujita, Halothane attenuates the cerebroprotective action of several Na+ and Ca2+ channel blockers via reversal of their ion channel blockade. Euro. J. Pharmacol. 452, 175-181 (2002).
111. M. Suwalsky et al., Dibucaine-induced modification of sodium transport in toad skin and of model membrane structures. Z. Naturforsch. C. J. Biosci. 56, 614-622 (2001).
112. C. Gutiérrez-Merino, A. Molina, B. Escudero, A. Diez, J. Laynez, Interaction of the local anesthetics dibucaine and tetracaine with sarcoplasmic reticulum membranes. Differential scanning calorimetry and fluorescence studies. Biochemistry 28, 3398-3406 (1989).
113. M. Volpi, R. I. Sha'afi, M. B. Feinstein, Antagonism of calmodulin by local anesthetics. Inhibition of calmodulin-stimulated calcium transport of erythrocyte inside-out membrane vesicles. Mol. Pharmacol. 20, 363-370 (1981).
114. M. A. Ramirez, N. L. Borja, Epalrestat: an aldose reductase inhibitor for the treatment of diabetic neuropathy. Pharmacotherapy 28, 646-655 (2008).
115. J. W. Steele, D. Faulds, K. L. Goa, Epalrestat. A review of its pharmacology, and therapeutic potential in late-onset complications of diabetes mellitus. Drugs Aging 3, 532-555 (1993).
116. L. P. Wakelin, M. J. Waring, Kinetics of drug-DNA interaction. Dependence of the binding mechanism on structure of the ligand. J. Mol. Biol. 144, 183-214 (1980).
117. R. F. Martin, T. R. Bradley, G. S. Hodgson, Cytotoxicity of an 125I-labeled DNA-binding compound that induces double-stranded DNA breaks. Cancer Res. 39, 3244-3247 (1979).
118. X. Cheng, Z. Yin, L. Rong, W. Hang, Subcellular chemical imaging of structurally similar acridine drugs by near-field laser desorption/laser postionization mass spectrometry. Nano Res. 13, 745-751 (2020).
119. Y. Fujii, K. Nonaka, S. Ryozawa, Use of probe-based confocal laser endomicroscopy for colon adenomas with topical application of acrinol drops. Dig. Endosc. 31, 101 (2019).
120. Y. Kumagai, K. Takubo, H. Ishida, Acrinol: Dye with potential for nuclear staining in confocal laser endomicroscopy. Dig. Endosc. 29, 811-812 (2017).
121. R. Berni, M. Clerici, G. Malpeli, L. Cleris, F. Formelli, Retinoids: in vitro interaction with retinol-binding protein and influence on plasma retinol. FASEB J. 7, 1179-1184 (1993).
122. H. Baldwin et al., 50 Years of Topical Retinoids for Acne: Evolution of Treatment. Am. J. Clin. Dermatol. 22, 315-327 (2021).
123. O. Nuñez, B. Chavez, R. Shaktah, P. P. Garcia, T. Minehan, Synthesis and DNA binding profile of monomeric, dimeric, and trimeric derivatives of crystal violet. Bioorg. Chem. 83, 297-302 (2019).
124. H. R. Arias, P. Bhumireddy, G. Spitzmaul, J. R. Trudell, C. Bouzat, Molecular mechanisms and binding site location for the noncompetitive antagonist crystal violet on nicotinic acetylcholine receptors. Biochemistry 45, 2014-2026 (2006).
125. C. S. Oliveira, R. Turchiello, A. J. Kowaltowski, G. L. Indig, M. S. Baptista, Major determinants of photoinduced cell death: Subcellular localization versus photosensitization efficiency. Free Radic. Biol. Med. 51, 824-833 (2011).
126. F. R. Gadelha, S. N. Moreno, W. De Souza, F. S. Cruz, R. Docampo, The mitochondrion of Trypanosoma cruzi is a target of crystal violet toxicity. Mol. Biochem. Parasitol. 34, 117-126 (1989).
127. A. M. Maley, J. L. Arbiser, Gentian violet: a 19th century drug re-emerges in the 21st century. Exp. Dermatol. 22, 775-780 (2013).
128. X. Zhang et al., Disruption of the mitochondrial thioredoxin system as a cell death mechanism of cationic triphenylmethanes. Free Radic. Biol. Med. 50, 811-820 (2011).
129. Y. Yang, D. Li, Investigation on the interaction between isorhamnetin and bovine liver catalase by spectroscopic techniques under different pH conditions. Luminescence 31, 1130-1137 (2016).
130. J. E. Kim et al., Isorhamnetin suppresses skin cancer through direct inhibition of MEK1 and PI3-K. Cancer Prev. Res. 4, 582-591 (2011).
131. Y. Zhang et al., Dietary component isorhamnetin is a PPARγ antagonist and ameliorates metabolic disorders induced by diet or leptin deficiency. Sci. Rep. 6, 19288 (2016).
132. G. Gong et al., Isorhamnetin: A review of pharmacological effects. Biomed. Pharmacother. 128, 110301 (2020).
133. H. Lei, Y. Qi, Z. G. Jia, W. L. Lin, Q. Wei, Studies on the interactions of kaempferol to calcineurin by spectroscopic methods and docking. Biochim. Biophys. Acta 1794, 1269-1275 (2009).
134. K. M. Lee et al., Phosphatidylinositol 3-kinase, a novel target molecule for the inhibitory effects of kaempferol on neoplastic cell transformation. Carcinogenesis 31, 1338-1343 (2010).
135. W. H. Hu et al., Kaempferol, a Major Flavonoid in Ginkgo Folium, Potentiates Angiogenic Functions in Cultured Endothelial Cells by Binding to Vascular Endothelial Growth Factor. Front. Pharmacol. 11, 526 (2020).
136. J. H. Kim et al., Kaempferol and Its Glycoside, Kaempferol 7-O-Rhamnoside, Inhibit PD-1/PD-L1 Interaction In Vitro. Int. J. Mol. Sci. 21, (2020).
137. H. S. Lee, G. S. Jeong, Therapeutic effect of kaempferol on atopic dermatitis by attenuation of T cell activity via interaction with multidrug resistance-associated protein 1. Br. J. Pharmacol. 178, 1772-1788 (2021).
138. M. H. Hoang et al., Kaempferol ameliorates symptoms of metabolic syndrome by regulating activities of liver X receptor-β. J. Nutr. Biochem. 26, 868-875 (2015).
139. J. Silva Dos Santos, J. P. Gonçalves Cirino, P. de Oliveira Carvalho, M. M. Ortega, The Pharmacological Action of Kaempferol in Central Nervous System Diseases: A Review. Front. Pharmacol. 11, 565700 (2020).
140. W. Alam, H. Khan, M. A. Shah, O. Cauli, L. Saso, Kaempferol as a Dietary Anti-Inflammatory Agent: Current Therapeutic Standing. Molecules 25, (2020).
141. S. K. Wong, K. Y. Chin, S. Ima-Nirwana, The Osteoprotective Effects Of Kaempferol: The Evidence From In Vivo And In Vitro Studies. Drug Des. Devel. Ther. 13, 3497-3514 (2019).
142. J. Ren et al., Recent progress regarding kaempferol for the treatment of various diseases. Exp. Ther. Med. 18, 2759-2776 (2019).
143. M. Imran et al., Kaempferol: A Key Emphasis to Its Anticancer Potential. Molecules 24, (2019).
144. Q. Wu, A. L. Tian, G. Kroemer, O. Kepp, Autophagy induction by IGFIR inhibition with picropodophyllin and linsitinib. Autophagy 17, 2046-2047 (2021).
145. T. Anastassiadis et al., A Highly Selective Dual Insulin Receptor (IR)/Insulin-like Growth Factor 1 Receptor (IGF-1R) Inhibitor Derived from an Extracellular Signal-regulated Kinase (ERK) Inhibitor. J. Biol. Chem. 288, 28068-28077 (2013).
146. Y. J. Wang, Y. K. Zhang, R. J. Kathawala, Z. S. Chen, Repositioning of Tyrosine Kinase Inhibitors as Antagonists of ATP-Binding Cassette Transporters in Anticancer Drug Resistance. Cancers 6, 1925-1952 (2014).
147. B. Ray et al., Deciphering molecular aspects of interaction between anticancer drug mitoxantrone and tRNA. J. Biomol. Struct. Dyn. 35, 2090-2102 (2017).
148. Z. Hajihassan, A. Rabbani-Chadegani, Studies on the binding affinity of anticancer drug mitoxantrone to chromatin, DNA and histone proteins. J. Biomed. Sci. 16, 31 (2009).
149. V. M. Golubovskaya et al., Mitoxantrone targets the ATP-binding site of FAK, binds the FAK kinase domain and decreases FAK, Pyk-2, c-Src, and IGF-1R in vitro kinase activities. Anticancer Agents Med. Chem. 13, 546-554 (2013).
150. X. Wan et al., A new target for an old drug: identifying mitoxantrone as a nanomolar inhibitor of PIM1 kinase via kinome-wide selectivity modeling. J. Med. Chem. 56, 2619-2629 (2013).
151. A. Feofanov, S. Sharonov, F. Fleury, I. Kudelina, I. Nabiev, Quantitative confocal spectral imaging analysis of mitoxantrone within living K562 cells: intracellular accumulation and distribution of monomers, aggregates, naphtoquinoxaline metabolite, and drug-target complexes. Biophys. J. 73, 3328-3336 (1997).
152. M. E. Fox, P. J. Smith, Subcellular localisation of the antitumour drug mitoxantrone and the induction of DNA damage in resistant and sensitive human colon carcinoma cells. Cancer Chemother. Pharmacol. 35, 403-410 (1995).
153. B. J. Evison, B. E. Sleebs, K. G. Watson, D. R. Phillips, S. M. Cutts, Mitoxantrone, More than Just Another Topoisomerase II Poison. Med. Res. Rev. 36, 248-299 (2016).
154. D. Matsuda et al., Molecular target of piperine in the inhibition of lipid droplet accumulation in macrophages. Biol. Pharm. Bull. 31, 1063-1066 (2008).
155. R. K. Reen, S. F. Roesch, F. Kiefer, F. J. Wiebel, J. Singh, Piperine impairs cytochrome P4501A1 activity by direct interaction with the enzyme and not by down regulation of CYP1A1 gene expression in the rat hepatoma 5 L cell line. Biochem. Biophys. Res. Commun. 218, 562-569 (1996).
156. D. Tolkatchev et al., Piperine, an alkaloid inhibiting the super-relaxed state of myosin, binds to the myosin regulatory light chain. Arch. Biochem. Biophys. 659, 75-84 (2018).
157. Z. Liu et al., Natural product piperine alleviates experimental allergic encephalomyelitis in mice by targeting dihydroorotate dehydrogenase. Biochem. Pharmacol. 177, 114000 (2020).
158. G. Zazeri, A. P. R. Povinelli, M. F. Lima, M. L. Cornélio, Detailed Characterization of the Cooperative Binding of Piperine with Heat Shock Protein 70 by Molecular Biophysical Approaches. Biomedicines 8, 629 (2020).
159. I. U. Haq et al., Piperine: A review of its biological effects. Phytother Res 35, 680-700 (2021).
160. W. D. Sasikala, A. Mukherjee, Intercalation and de-intercalation pathway of proflavine through the minor and major grooves of DNA: roles of water and entropy. Phys. Chem. Chem. Phys. 15, 6446-6455 (2013).
161. R. Sinha, M. Hossain, G. S. Kumar, Interaction of Small Molecules with Double-Stranded RNA: Spectroscopic, Viscometric, and calorimetric Study of Hoechst and Proflavine Binding to PolyCG Structures. DNA Cell Biol. 28, 209-219 (2009).
162. E. S. DeJong, C.-e. Chang, M. K. Gilson, J. P. Marino, Proflavine Acts as a Rev Inhibitor by Targeting the High-Affinity Rev Binding Site of the Rev Responsive Element of HIV-1. Biochemistry 42, 8035-8046 (2003).
163. J. Diekmann et al., The Photoaddition of a Psoralen to DNA Proceeds via the Triplet State. J. Am. Chem. Soc. 141, 13643-13653 (2019).
164. W. Xia et al., Photo-Activated Psoralen Binds the ErbB2 Catalytic Kinase Domain, Blocking ErbB2 Signaling and Triggering Tumor Cell Apoptosis. PLOS One 9, e88983 (2014).
165. Z. Lu et al., RNA Duplex Map in Living Cells Reveals Higher-Order Transcriptome Structure. Cell 165, 1267-1279 (2016).
166. C. D. Fitch, Ferriprotoporphyrin IX, phospholipids, and the antimalarial actions of quinoline drugs. Life Sci. 74, 1957-1972 (2004).
167. S. Slavkovic, Z. R. Churcher, P. E. Johnson, Nanomolar binding affinity of quinine-based antimalarial compounds by the cocaine-binding aptamer. Bioorg. Med. Chem. 26, 5427-5434 (2018).
168. S. Kobayashi et al., The specificity of inhibition of debrisoquine 4-hydroxylase activity by quinidine and quinine in the rat is the inverse of that in man. Biochem. Pharmacol. 38, 2795-2799 (1989).
169. M. Dziekan Jerzy et al., Identifying purine nucleoside phosphorylase as the target of quinine using cellular thermal shift assay. Sci. Trans. Med. 11, eaau3174 (2019).
170. Z. Wang et al., Pyrroloquinoline quinine protects HK-2 cells against high glucose-induced oxidative stress and apoptosis through Sirt3 and PI3K/Akt/Fox03a signaling pathway. Biochem. Biophys. Res. Commun. 508, 398-404 (2019).
171. M. Carlquist, T. Frejd, M. F. Gorwa-Grauslund, Flavonoids as inhibitors of human carbonyl reductase 1. Chem. Biol. Interact. 174, 98-108 (2008).
172. L. S. Chua, A review on plant-based rutin extraction methods and its pharmacological activities. J. Ethnopharmacol. 150, 805-817 (2013).
173. K. Horvathova, L. Novotný, D. Tothova, A. Vachalkova, Determination of free radical scavenging activity of quercetin, rutin, luteolin and apigenin in H2O2-treated human ML cells K562. Neoplasma 51, 395-399 (2004).
174. F. Liu et al., Scutellarin Suppresses Patient-Derived Xenograft Tumor Growth by Directly Targeting AKT in Esophageal Squamous Cell Carcinoma. Cancer Prev. Res. 12, 849-860 (2019).
175. J. Dai et al., Scutellarin protects the kidney from ischemia/reperfusion injury by targeting Nrf2. Nephrology.
176. L. Wang, Q. Ma, Clinical benefits and pharmacology of scutellarin: a comprehensive review. Pharmacol. Therapeut. 190, 105-127 (2018).
177. Å. Rosenquist et al., Discovery and Development of Simeprevir (TMC435), a HCV NS3/4A Protease Inhibitor. J. Med. Chem. 57, 1673-1693 (2014).
178. A. Kohli, A. Shaffer, A. Sherman, S. Kottilil, Treatment of Hepatitis C: A Systematic Review. JAMA 312, 631-640 (2014).
179. G. S. Papaetis, K. N. Syrigos, Sunitinib. BioDrugs 23, 377-389 (2009).
180. S. Hu et al., Interaction of the Multikinase Inhibitors Sorafenib and Sunitinib with Solute Carriers and ATP-Binding Cassette Transporters. Clin. Cancer Res. 15, 6062-6069 (2009).
181. R. Fröbom et al., Direct interaction of the ATP-sensitive K+ channel by the tyrosine kinase inhibitors imatinib, sunitinib and nilotinib. Biochem. Biophys. Res. Commun. 557, 14-19 (2021).
182. N. Andrae et al., Sunitinib targets PDGF-receptor and Flt3 and reduces survival and migration of human meningioma cells. Eur. J. Cancer 48, 1831-1841 (2012).
183. J. J. W. Wong et al., Photochemically-Induced Release of Lysosomal Sequestered Sunitinib: Obstacles for Therapeutic Efficacy. Cancers 12, 417-417 (2020).
184. R. J. Honeywell, S. M. Hitzerd, G. A. M. Kathmann, G. J. Peters, Subcellular localization of several structurally different tyrosine kinase inhibitors. ADMET DMPK 6, 258-266 (2018).
185. L. Q. M. Chow, S. G. Eckhardt, Sunitinib: From Rational Design to Clinical Efficacy. J. Clin. Oncol. 25, 884-896 (2007).
186. N. Wiedemar, A. Hauser Dennis, P. Mäser, 100 Years of Suramin. Antimicrob. Agents Chemther. 64, e01168-01119 (2020).
187. Z. Jiang, W. Gao, L. Huang, Tanshinones, Critical Pharmacological Components in Salvia miltiorrhiza. Front. Pharmacol. 10, 202 (2019).
188. C. Zhong et al., Recent Research Progress (2015-2021) and Perspectives on the Pharmacological Effects and Mechanisms of Tanshinone IIA. Front. Pharmacol. 12, (2021).
189. K.-W. Jeong et al., Dynamics of a Heparin-Binding Domain of VEGF165 Complexed with Its Inhibitor Triamterene. Biochemistry 50, 4843-4854 (2011).
190. H. Knauf, U. Wais, R. Lübcke, G. Albiez, On the Mechanism of Action of Triamterene: Effects on Transport of Na+, K+, and HVHCO-3-ions. Eur. J. Clin. Invest. 6, 43-50 (1976).
191. E. Noack, P. Schuhmacher, The interaction of triamterene at the myocardial beta receptor site. J. Mol. Cell. Cardiol. 15, 319-324 (1983).
192. V. D. Wiebelhaus et al., The diuretic and natruretic activity of triamterene and several related pteridinesin the rat. J. Pharmacol. Exp. Ther. 149, 397 (1965).
193. D. A. Koster et al., Single-molecule observations of topotecan-mediated TopIB activity at a unique DNA sequence. Nucl. Acids. Res. 36, 2301-2310 (2008).
194. W. Bocian et al., Binding of topotecan to a nicked DNA oligomer in solution. Eur. J. Chem. 14, 2788-2794 (2008).
195. M. Kobori et al., Wedelolactone suppresses LPS-induced caspase-11 expression by directly inhibiting the IKK Complex. Cell Death Differ. 11, 123-130 (2004).
196. T. Kučírková et al., Anti-cancer effects of wedelolactone: interactions with copper and subcellular localization. Metallomics 10, 1524-1531 (2018).
197. M.-m. Zhu et al., Wedelolactone alleviates doxorubicin-induced inflammation and oxidative stress damage of podocytes by IκK/IκB/NF-κB pathway. Biomed. Pharmacother. 117, 109088 (2019).
198. F. Ali, B. A. Khan, S. Sultana, Wedelolactone mitigates UVB induced oxidative stress, inflammation and early tumor promotion events in murine skin: plausible role of NFkB pathway. Eur. J. Pharmacol. 786, 253-264 (2016).
199. E. Yao et al., Phytochemical wedelolactone reverses obesity by prompting adipose browning through SIRT1/AMPK/PPARα pathway via targeting nicotinamide N-methyltransferase. Phytomedicine 94, 153843 (2022).
200. K. Harkin, J. Augustine, A. W. Stitt, H. Xu, M. Chen, Wedelolactone Attenuates Nmethyl-N-nitrosourea-Induced Retinal Neurodegeneration through Suppression of the AIM2/CASP11 Pathway. Biomedicines 10, (2022).
201. J.-y. Yang et al., Wedelolactone Attenuates Pulmonary Fibrosis Partly Through Activating AMPK and Regulating Raf-MAPKs Signaling Pathway. Front. Pharmacol. 10, (2019).
202. N. Romanchikova, P. Trapencieris, Wedelolactone Targets EZH2-mediated Histone H3K27 Methylation in Mantle Cell Lymphoma. Anticancer Res. 39, 4179-4184 (2019).
203. P. Yu et al., Characterization of the Activity of the PI3K/mTOR Inhibitor XL765 (SAR245409) in Tumor Models with Diverse Genetic Alterations Affecting the PI3K Pathway. Mol. Cancer Therapeut. 13, 1078-1091 (2014).
204. K. P. Papadopoulos et al., Phase I Safety, Pharmacokinetic, and Pharmacodynamic Study of SAR245409 (XL765), a Novel, Orally Administered PI3K/mTOR Inhibitor in Patients with Advanced Solid Tumors. Clin. Cancer Res. 20, 2445-2456 (2014).

### Proteins and Corresponding Condensates

Table 1 lists proteins and corresponding condensates suitable for use with the methods and systems described herein. In some embodiments, the condensate is a condensate found within cells of a mammal. In some embodiments, the condensate is associated with cells of a particular disease. In some embodiments, the condensate is a condensate of a model organism, which is useful for research purposes.

TABLE 1
Proteins and corresponding condensates

| | Scaffold | | |
|---|---|---|---|
| Protein Name(s) | protein | Condensate | Reference(s) |
| Nucleophosmin 1 | NPM1 | Nucleolus granular | (1) |
| | | cluster | |
| mediator of RNA pol II | MED1 | Transcriptional | (1) |
| transcription subunit I; | | condensate | |
| Mediator complex subunit 1 | | | |
| Heterochromatin 1-alpha | HP1α | Heterochromatin | (1) |
| Serine and arginine splicing | SRSF2 | Splicing condensate | (2) |
| factor 2 | | | |
| fused in sarcoma | FUS | Paraspeckle | (3) |
| Nucleocapsid protein N | SARS- | Viral replication- | (4-7) |
| | CoV-2 | transcription complex | |
| | Nucleocapsid | | |
| RAS GTPase-activation | G3BP1 | Stress granule | (8) |
| protein-binding protein1, | | | |
| G3BP1 stress granule assemble | | | |
| factor 1 | | | |
| Small ubiquitin modifier, | SUMO/Sim | PML nuclear bodies | (9) |
| single-minded homolog 1 | | | |
| SRC homology 3 domain | SH3 | Signaling | (10) |
| TAR DNA binding protein 43 | TDP-43 | RNP granules | (11, 12) |
| Deadbox helicases 4, 6 | DDX4 | Germ granules | (13) |
| chromobox protein homolog 2 | CBX2 | Polycomb body | (14) |
| Fibirilin 1 | FIB1 | Nucleolus fibrillar | (15) |
| | | cluster | |
| respirator syncytial virus | RSV N/P | Viral inclusion bodies | (16) |
| nucleocapsid protein | | | |
| methyl CpG binding protein 2 | MeCP2 | Heterochromatin | (17) |
| bromodomain 4 | BRD4 | Transcriptional | (18) |
| | | condensate | |
| Receptor tyrosine kinases | RTKs | Signaling condensate | (19) |
| (various) | | | |
| cyclic GMP-AMP synthase, | cGAS- | Signaling condensate | (20, 21) |
| Stimulator of interferon genes | STING | | |
| Early flowering 3 | ELF3 | Thermal sensor | (22) |
| Neural Wiskott-Aldrich | N-WASP | Cytoskeleton | (23) |
| syndrome protein | | | |
| tumor surpressor p53-binding | 53BP1/p53 | DNA damage and | (24) |
| protein 1, tumor protein p53 | | repair | |
| spindle defective protein 5 | SPD-5 | Cytoskeleton | (25) |
| carboxysome assembly protein | CcmM | Beta-carboxysome | (26) |
| | | biogenesis | |
| miRNA induced silencing | miRISC | Deadenylation | (27) |
| complex | | | |
| psohporibosylformylglycinamide | FGAMS | Purinosome, Purine | (28) |
| | | biosynthesis | |
| K63, heterogeneous nuclear | P-body: | P-BODY | (29) |
| ribonucleoplrotein U, Insulin | K63, | | |
| like growth factor 2 mRNA | HNRNPU, | | |
| binding protein 1, DExH-box | IGF2BP1, | | |
| helicase 9, Insulin like growth | DHX9, | | |
| factor 2 mRNA binding protein | IG2BP3, | | |
| 3, | SYNCRIP | | |
| Synaptotagmin Binding | | | |
| Cytoplasmic RNA Interacting | | | |
| Protein, | | | |
| Deadbox helicase homolog 1 | dhh1 | Uridine rich snRNP | (30) |
| | | bdoy | |
| RNA binding protein, mRNA | Rbpms2 | Balbiani body | (31, 32) |
| processing factor 2 | | | |
| protein component of C. | PG11/PGL3 | P-granule/chromatoid | (33) |
| elegeans germ granules 1 and 3 | | body | |
| heterogeneous nuclear | hnRNPA2 | RNA transport | (34) |
| ribonucleoproteins A2 | | granules | |
| nucleoporin 49 | NUP49, | Nuclera pore complex | (35) |
| nucleoporin 89 | NUP89 | | |
| Coilin | Coilin | Cajal body | (36) |
| Survival motor neuron | SMN | Gemini of cajal bodies | (37) |
| Serine/Arginine-rich splicing | SC35 | Nuclear speckles | (38) |
| factor 35 | | | |
| hnRNP I | SH54 PTB | Perinucleolar | (39) |
| | | compartment | |
| PML protein | PML | PML body | (40) |
| | Protein | | |
| U7 | U7 | Histone locus body | (41) |
| Crk-associated substrate | p130cas/ | Adhesion clusters | (42) |
| focal adhesion kinase | FAK | | |
| Linker for activation of T cells | LAT | T-cell activation | (43) |
| | clusters | | |
| Annexxin 11 | Annexxin | Cytokinesis | (44) |
| | 11/A11 | | |
| Large protein 1 | lge1/bre1 | Gene body histone | (45) |
| E3 ubiquiting ligase bre1 | | ubquitination | |
| Transcriptional repressor | CTCF | CTCF clusters | (46) |
| CTCF | | | |
| Yes associated protein | YAP | Osomotic stress | (47) |
| speckle type POZ protein | SPOP/DAXX | Protein ubiquitination | (49) |
| death domain-associated | | | |
| protein 6 | | | |
| chromobox protein homolog 2 | CBX2 | PcG bodies | (50) |
| origin recognition complex | ORC, | Specification of DNA | (50) |
| cell division cycle 6 | Cdc6, cdt1 | origins | |
| Chromatin licensing and DNA | | | |
| replication factor 1 | | | |
| CXXC repeat containing | CRIPT | PDZ domain | |
| interactor of PDZ3 domain | | interactions | |
| 2-C-methyl-D-erythritol 4- | ISPD | O-linked | |
| phosphate cytidylyltransferase | | mannosylation | |
| Diacylglycerol O- | DGAT1 | Diacylglycerol O- | |
| Acyltransferase 1 | | acyltransferase | |
| carnitine palmitoyltransferase 2 | CPT2 | Carnitine | |
| | | palmitoyltransferase 2 | |
| exostosin like | EXTL2 | Gylcosyl transferase | |
| glycosyltransferase 2 | | | |
| polypeptide N- | GALNT5 | Acetylgalactosaminyltransferase | |
| acetylgalactosaminyltransferase 5 | | | |
| sulfotransferase family1b | SULT1b1 | Sulfate transferase | |
| member 1 | | | |
| poly(ADP-ribose) polymerase | PARP10 | Mono-ADP | |
| family member 10 | | ribosylation | |
| phosphatidylinositol glycan | PIGO | Ethanolamine | |
| anchor biosynthesis class O | | phosphate transferase | |
| Deoxynucleotidyltransferase | DNTTIP1 | DNTT terminal | |
| terminal interacting protein 1 | | deoxynucleotidyltransferase | |
| activin a receptor type 1 | ACVR1 | Ser/Thr kinase | |
| Xylulokinase | XYLB | D-xylulose | |
| | | phosphorylation | |
| 3′-Phosphoadenosine 5′- | PAPSS1 | ATP sulfurylase/APS | |
| Phosphosulfate Synthase 1 | | kinase | |
| trans-golgi network integral | TGN46 | Trans-golgi network | (51) |
| membrane protein 2 | | | |
| RAP guanine nucleotide | Epac1 | CAMP regulated | (52) |
| exchange factor 3 | | sumyolation | |
| Exchange factor directly | | | |
| activated by cAMP 1 | | | |
| Nup89 | Oncogenic | | (53) |
| | fusion | | |
| | proteins | | |
| VPS41 subunit of HOPS | VPS41 | Vegetative growth and | (54) |
| complex | | vacuolar transport | |
| Endocytic adaptor | Eps15/Ede1 | Endocytosis initiation | (55) |
| Mal, T cell differentiation | MALL | Overexpressed | (56) |
| protein like | | proteolipid in cancer | |
| nuclear receptor coactivator 4 | NCOA4 | Iron homeostasis | (57) |
| Arabidopsis EH protein 1 | AtEH/Pan1 | Clathrin mediated | (58) |
| | | endocytosis | |
| cip1-interacting zinc finger | CIZ1 | X-chromosome | (59) |
| protein | | assembly | |
| Ul112-113 | UL112-113 | Human | (60) |
| | | cytomegalovirus | |
| post synaptic density protein 95, | PSD- | Post synaptic density | (61) |
| synaptic RAS GTPase | 95/SynGAP | | |
| activating protein 1 | | | |
| Tight junction protein 1 | ZO-1 | Tight junction protein | (62) |
| Sequestrome 1 | p62 | Proteasomal | (63) |
| | | degradation | |

### References for Table 1

1. I. A. Klein, e. al., Partitioning of cancer therapeutics in nuclear condensates. Science 368, 1386 (2020).
2. Y. E. Guo, e. al., Pol II phosphorylation regulates a switch between transcriptional and splicing condensates. Nature 572, 543-548 (2019).
3. J. Wang, e. al., A molecular grammar governing the driving forces for phase separation of prion-like RNA binding proteins Cell 174, 688-699.e616 (2018).
4. S. Wang, e. al., Targeting liquid-liquid phase separation of SARS-COV-2 nucleocapsid protein promotes innate antiviral immunity by elevating MAVS activity. Nat. Cell. Biol. 23, 718-732 (2021).
5. A. Savastano, A. Ibáñez de Opakua, M. Rankovic, M. Zweckstetter, Nucleocapsid protein of SARS-COV-2 phase separates into RNA-rich polymerase-containing condensates. Nat. Commun. 11, 6041 (2020).
6. S. Lu, e. al., The SARS-COV-2 nucleocapsid phosphoprotein forms mutually exclusive condensates with RNA and the membrane-associated M protein. Nat. Commun. 12, 502 (2021).
7. J. Cubuk et al., The SARS-COV-2 nucleocapsid protein is dynamic, disordered, and phase separates with RNA. Nature Communications 12, 1936 (2021).
8. P. Yang et al., G3BP1 Is a Tunable Switch that Triggers Phase Separation to Assemble Stress Granules. Cell 181, 325-345.e328 (2020).
9. S. F. Banani, e. al., Composition control of phase-separated bodies. Cell 166, 651-663 (2016).
10. K. Hong, D. Song, Y. Jung, Behavior control of membrane-less protein liquid condensates with metal ion-induced phase separation. Nature Communications 11, 5554 (2020).
11. G. Krainer, e. al., Reentrant liquid condensate phase of proteins is stabilized by hydrophobic and non-ionic interactions. Nat. Commun. 12, 1085 (2021).
12. M. Y. Fang, e. al., Small-molecule modulation of TDP-43 recruitment to stress granules prevents persistent TDP-43 accumulation in ALS/FTD. Neuron 103, 802-819.e811 (2019).
13. T. J. Nott, e. al., Phase transition of a disordered nuage protein generates environmentally responsive membraneless organelles. Mol. Cell 57, 936-947 (2015).
14. R. Tatavosian et al., Nuclear condensates of the Polycomb protein chromobox 2 (CBX2) assemble through phase separation. J Biol Chem 294, 1451-1463 (2019).
15. M. Feric, T. Misteli, Phase separation in genome organization across evolution. Trends Cell Biol. 31, 671-685 (2021).
16. J. Risso-Ballester, e. al., A condensate-hardening drug blocks RSV replication in vivo. Nature 595, 596-599 (2021).
17. C. H. Li et al., MeCP2 links heterochromatin condensates and neurodevelopmental disease. Nature 586, 440-444 (2020).
18. B. R. Sabari, e. al., Coactivator condensation at super-enhancers links phase separation and gene control. Science 361, eaar3958 (2018).
19. C.-C. Lin et al., Receptor tyrosine kinases regulate signal transduction through a liquid-liquid phase separated state. Molecular Cell 82, 1089-1106.e1012 (2022).
20. M. Du, J. Chen Zhijian, DNA-induced liquid phase condensation of cGAS activates innate immune signaling. Science 361, 704-709 (2018).
21. P. J. Flynn, P. D. Koch, T. J. Mitchison, Chromatin bridges, not micronuclei, activate cGAS after drug-induced mitotic errors in human cells. Proceedings of the National Academy of Sciences 118, e2103585118 (2021).
22. J.-H. Jung et al., A prion-like domain in ELF3 functions as a thermosensor in Arabidopsis. Nature 585, 256-260 (2020).
23. L. B. Case, X. Zhang, J. A. Ditlev, M. K. Rosen, Stoichiometry controls activity of phase-separated clusters of actin signaling proteins. Science 363, 1093-1097 (2019).
24. O. Karni-Schmidt et al., Energy-dependent nucleolar localization of p53 in vitro requires two discrete regions within the p53 carboxyl terminus. Oncogene 26, 3878-3891 (2007).
25. J. B. Woodruff et al., The Centrosome Is a Selective Condensate that Nucleates Microtubules by Concentrating Tubulin. Cell 169, 1066-1077.e1010 (2017).
26. H. Wang et al., Rubisco condensate formation by CcmM in β-carboxysome biogenesis. Nature 566, 131-135 (2019).
27. J. Sheu-Gruttadauria, I. J. MacRae, Phase Transitions in the Assembly and Function of Human miRISC. Cell 173, 946-957.e916 (2018).
28. A. M. Pedley et al., Purine biosynthetic enzymes assemble into liquid-like condensates dependent on the activity of chaperone protein HSP90. Journal of Biological Chemistry 298, 101845 (2022).
29. Y. Luo, Z. Na, S. A. Slavoff, P-Bodies: Composition, Properties, and Functions. Biochemistry 57, 2424-2431 (2018).
30. K. Weis, Dead or alive: DEAD-box ATPases as regulators of ribonucleoprotein complex condensation. Biological Chemistry 402, 653-661 (2021).
31. J. W. Schneider et al., Dysregulated ribonucleoprotein granules promote cardiomyopathy in RBM20 gene-edited pigs. Nature Medicine 26, 1788-1800 (2020).
32. O. H. Kaufman, K. Lee, M. Martin, S. Rothhämel, F. L. Marlow, rbpms2 functions in Balbiani body architecture and ovary fate. PLOS Genetics 14, e1007489 (2018).
33. H. Dannenberg, P. Komminoth, W. N. M. Dinjens, E. J. M. Speel, R. R. de Krijger, Molecular genetic alterations in adrenal and extra-adrenal pheochromocytomas and paragangliomas. Endocrine Pathology 14, 329-350 (2003).
34. D. Tauber, G. Tauber, R. Parker, Mechanisms and Regulation of RNA Condensation in RNP Granule Formation. Trends Biochem Sci 45, 764-778 (2020).
35. G. Celetti et al., The liquid state of FG-nucleoporins mimics permeability barrier properties of nuclear pore complexes. Journal of Cell Biology 219, e201907157 (2019).
36. S. C. Ogg, A. I. Lamond, Cajal bodies and coilin-moving towards function. Journal of Cell Biology 159, 17-21 (2002).
37. V. Setola et al., Axonal-SMN (a-SMN), a protein isoform of the survival motor neuron gene, is specifically involved in axonogenesis. Proceedings of the National Academy of Sciences 104, 1959-1964 (2007).
38. M. Schilling et al., TOR signaling regulates liquid phase separation of the SMN complex governing snRNP biogenesis. Cell Reports 35, 109277 (2021).
39. C. Pollock, S. Huang, The Perinucleolar Compartment. Cold Spring Harbor Perspectives in Biology 2, (2010).
40. V. Lallemand-Breitenbach, PML nuclear bodies. Cold Spring Harbor perspectives in biology 2, a000661 (2010).
41. J.-L. Liu, G. Gall Joseph, U bodies are cytoplasmic structures that contain uridine-rich small nuclear ribonucleoproteins and associate with P bodies. Proceedings of the National Academy of Sciences 104, 11655-11659 (2007).
42. L. B. Case, M. De Pasquale, L. Henry, M. K. Rosen, Synergistic phase separation of two pathways promotes integrin clustering and nascent adhesion formation. eLife 11, e72588 (2022).
43. X. Su et al., Phase separation of signaling molecules promotes T cell receptor signal transduction. Science 352, 595-599 (2016).
44. P. A. G. Lillebostad et al., Structure of the ALS Mutation Target Annexin A11 Reveals a Stabilising N-Terminal Segment. Biomolecules 10, (2020).
45. L. D. Gallego et al., Phase separation directs ubiquitination of gene-body nucleosomes. Nature 579, 592-597 (2020).
46. A. S. Hansen, A. Amitai, C. Cattoglio, R. Tjian, X. Darzacq, Guided nuclear exploration increases CTCF target search efficiency. Nature Chemical Biology 16, 257-266 (2020).
47. D. Cai et al., Phase separation of YAP reorganizes genome topology for long-term YAP target gene expression. Nature Cell Biology 21, 1578-1589 (2019).
48. A. Pandya-Jones et al., A protein assembly mediates Xist localization and gene silencing. Nature 587, 145-151 (2020).
49. J. J. Bouchard et al., Cancer Mutations of the Tumor Suppressor SPOP Disrupt the Formation of Active, Phase-Separated Compartments. Molecular Cell 72, 19-36.e18 (2018).
50. R. Tatavosian et al., Nuclear condensates of the Polycomb protein chromobox 2 (CBX2) assemble through phase separation. Journal of Biological Chemistry 294, 1451-1463 (2019).
51. P. Lujan et al., Sorting of secretory proteins at the trans-Golgi network by TGN46. bioRxiv, 2022.2004.2020.488883 (2022).
52. W. Yang et al., Epac1 activation by cAMP regulates cellular SUMOylation and promotes the formation of biomolecular condensates. Science Advances 8, eabm2960.
53. I. Y. Quiroga, J. H. Ahn, G. G. Wang, D. Phanstiel, Oncogenic fusion proteins and their role in three-dimensional chromatin structure, phase separation, and cancer. Current Opinion in Genetics & Development 74, 101901 (2022).
54. D. Jiang et al., Arabidopsis HOPS subunit VPS41 carries out plant-specific roles in vacuolar transport and vegetative growth. Plant Physiology, kiac167 (2022).
55. M. Kozak, M. Kaksonen, Phase separation of Edel promotes the initiation of endocytic events. bioRxiv, 861203 (2019).
56. A. Rubio-Ramos et al., MALL, a membrane-tetra-spanning proteolipid overexpressed in cancer, is present in membraneless nuclear biomolecular condensates. Cellular and Molecular Life Sciences 79, 236 (2022).
57. S. Kuno, H. Fujita, Y.-k. Tanaka, Y. Ogra, K. Iwai, Iron-induced NCOA4 condensation regulates ferritin fate and iron homeostasis. EMBO reports n/a, e54278 (2022).
58. J. M. Dragwidge et al., AtEH/Pan1 proteins drive phase separation of the TPLATE complex and clathrin polymerisation during plant endocytosis. bioRxiv, 2022.2003.2017.484738 (2022).
59. S. Sofi et al., Prion-like domains drive CIZ1 assembly formation at the inactive X chromosome. Journal of Cell Biology 221, e202103185 (2022).
60. E. Caragliano et al., Human cytomegalovirus forms phase-separated compartments at viral genomes to facilitate viral replication. Cell Reports 38, 110469 (2022).
61. N. R. Christensen et al., Bidirectional protein-protein interactions control liquid-liquid phase separation of PSD-95 and its interaction partners. iScience 25, 103808 (2022).
62. N. Kinoshita et al., Force-dependent remodeling of cytoplasmic ZO-1 condensates contributes to cell-cell adhesion through enhancing tight junctions. iScience 25, 103846 (2022).
63. P. Erdbrügger, F. Wilfling, p62 condensates are a hub for proteasome-mediated protein turnover in the nucleus. Proceedings of the National Academy of Sciences 118, e2113647118 (2021).

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A computer-implemented method of quantifying partitioning of one or more test agents in an in vivo condensate, the method comprising:
a) training a machine-learning classifier on a training dataset, the training dataset comprising (i) a quantification of partitioning of training agents in an in vitro protein condensate that corresponds to the in vivo condensate and (ii) a representation of the training agents; and
b) applying a test dataset comprising a representation of the one or more test agents to the machine-learning classifier to quantify partitioning of the one or more test agents in the in vivo condensate.

2. The method of claim 1, wherein the machine-learning classifier is a random forest classifier.

3. The method of claim 1, wherein the machine-learning classifier is a message passing neural network.

4. The method of claim 1, wherein the message-passing neural network is a directed message-passing neural network.

5. The method of any one of claims 1 through 4, wherein training the machine-learning classifier further includes training a first machine-learning classifier on the training dataset, and training a second machine-learning classifier on the training dataset, and wherein applying the test dataset comprising the representation of the one or more test agents to the machine learning-classifier further includes applying the test dataset comprising the representation of the one or more test agents to the first machine-learning classifier and the second machine-learning classifier, thereby producing results from each respectively, and the method further comprises:
aggregating the respective results of the first machine-learning classifier and the second machine-learning classifier to quantify partitioning of the one or more test agents in the in vivo condensate.

6. The method of claim 5, wherein aggregating the respective results comprises:
determining whether the result of the first machine-learning classifier and the second machine-learning classifier indicate that a partitioning ratio of the one or more test agents exceed specified probability thresholds for the first machine-learning classifier and the second machine-learning classifier; and
if both of the respective results exceed the specified probability thresholds, quantifying the partitioning of the one or more test agents in the in vivo condensate based on the partitioning ratio.

7. The method of any one of claims 1 through 4, wherein the machine-learning classifier is one or more of a neural network, an artificial neural network, a graph neural network, a sequence neural network, a binary classifier, a forest classifier, a random forest classifier, and a message passing neural network.

8. The method of any one of claims 1 through 4, further comprising providing the training dataset.

9. The method of any one of claims 1 through 4, wherein the quantification of partitioning of training agents in the in vitro protein condensate is a partition ratio of a quantification of the training agents within the in vitro protein condensate versus a quantification of the training agents outside the in vitro protein condensate.

10. The method of any one of claims 1 through 4, wherein training the message-passing neural network comprises associating the representation of the training agents with one or more partition ratios in one or more condensates.

11. The method of any one of claims 1 through 4, wherein the representation of the one or more test agents and training agents is a representation of chemical structure.

12. The method of any one of claims 1 through 4, wherein the representation of the one or more test agents and training agents is a simplified molecular-input line-entry system (SMILES) representation of chemical structure.

13. The method of any one of claims 1 through 4, wherein the representation of the one or more test agents and training agents is a Morgan fingerprint of chemical structure.

14. The method of any one of claims 1 through 4, wherein the representation of the one or more test agents and training agents comprises chemical properties.

15. The method of claim 14, wherein the chemical properties are a vector comprising chemical property data.

16. The method of any one of claims 1 through 4, further comprising selecting a threshold for solvation, wherein the quantified partitioning of the one or more test agents in the in vivo condensate above the threshold indicates that the one or more test agents solvate in the in vivo condensate.

17. The method of any one of claims 1 through 4, further comprising applying a validation dataset comprising a representation of one or more validation agents to the machine-learning classifier.

18. The method of any one of claims 1 through 4, further comprising comparing a quantified partitioning of the one or more test agents in a first in vivo condensate to a quantified partitioning of the one or more test agents in a second in vivo condensate.

19. The method of any one of claims 1 through 4, wherein the in vitro protein condensate comprises a condensate selected from Table 1.

20. The method of any one of claims 1 through 4, wherein the in vivo protein condensate comprises a condensate selected from Table 1.

21. The method of any one of claims 1 through 4, wherein the in vitro protein condensate comprises MED1.

22. The method of any one of claims 1 through 4, wherein the in vitro protein condensate comprises NPM1.

23. The method of any one of claims 1 through 4, wherein the in vitro protein condensate comprises HP1α.

24. The method of any one of claims 1 through 4, wherein the in vivo protein condensate comprises MED1.

25. The method of any one of claims 1 through 4, wherein the in vivo protein condensate comprises NPM1.

26. The method of any one of claims 1 through 4, wherein the in vivo protein condensate comprises HP1α.

27. The method of any one of claims 1 through 4, wherein the one or more test agents comprise at least one of a small molecule, an RNA, an siRNA, a peptide, and a candidate therapeutic agent.

28. The method of any one of claims 1 through 4, further comprising selecting a test agent based on the quantified partitioning of the test agent in the in vivo condensate.

29. The method of claim 28, wherein the quantified partitioning of the selected test agent in the in vivo condensate is greater than or equal to a selected threshold for solvation.

30. The method of claim 28, wherein the quantified partitioning of the selected test agent in the in vivo condensate is less than or equal to a selected threshold for solvation.

31. The method of claim 28, further comprising administering the selected test agent to cells to determine in vivo partitioning of the test agent.

32. The method of any one of claims 1 through 4, further comprising repeating a) and b) for a plurality of in vitro protein condensates for a corresponding plurality of in vivo condensates.

33. The method of claim 32, further comprising comparing the quantified partitioning of the one or more test agents in the plurality of in vivo condensates.

34. The method of claim 33, further comprising selecting a test agent based on relative partitioning of the test agent into the plurality of in vivo condensates.

35. The method of claim 34, further comprising administering the selected test agent to cells to determine in vivo partitioning of the selected test agent into the plurality of in vivo condensates.

36. The method of any one of claims 1 through 4, wherein the in vivo condensate comprises a biological target of the selected test agent.

37. The method of any one of claims 1 through 4, further comprising generating the training dataset by:
a) forming an in vitro condensate of a protein;
b) administering training agents to the condensate;
c) detecting a signal inside the condensate and signal outside the condensate;
d) determining a partition ratio of the signal inside the condensate divided by the signal outside the condensate; and
e) repeating a) through d) for a plurality of training agents to generate the training dataset.

38. The method of claim 37, wherein the protein of the in vitro condensate is fused to a tag.

39. The method of claim 38, wherein the tag is a fluorescent protein, and wherein detecting the signal comprises detecting a fluorescent signal.

40. A method of quantifying partitioning of one or more test agents in an in vivo condensate, the method comprising:
a) applying a test dataset comprising a representation of the one or more test agents to a machine-learning classifier to quantify partitioning of the one or more test agents in the in vivo condensate, the machine-learning classifier trained on a training dataset that comprises (i) a quantification of partitioning of training agents in an in vitro protein condensate that corresponds to the in vivo condensate and (ii) a representation of one or more training agents.

41. The method of claim 40, wherein the machine learning algorithm is a random forest classifier.

42. The method of claim 40, wherein the machine learning algorithm is a message-passing neural network.

43. A system for quantifying partitioning of one or more test agents in an in vivo condensate, the system comprising:
a processor; and
a memory with computer code instructions stored thereon, the processor and the memory, with the computer code instructions, being configured to cause the system to:
a) train a machine-learning classifier on a training dataset, the training dataset comprising (i) a quantification of partitioning of training agents in an in vitro protein condensate that corresponds to the in vivo condensate and (ii) a representation of the training agents; and
b) apply a test dataset comprising a representation of the one or more test agents to the machine-learning classifier to quantify partitioning of the one or more test agents in the in vivo condensate.

44. A non-transitory computer readable medium with instructions stored thereon for quantifying partitioning of one or more test agents in an in vivo condensate, the instructions, when executed by a processor, causing the processor to:
a) train a machine-learning classifier on a training dataset, the training dataset comprising (i) a quantification of partitioning of training agents in an in vitro protein condensate that corresponds to the in vivo condensate and (ii) a representation of the training agents; and
b) apply a test dataset comprising a representation of the one or more test agents to the machine-learning classifier to quantify partitioning of the one or more test agents in the in vivo condensate.

45. A system for quantifying partitioning of one or more test agents in an in vivo condensate, the system comprising:
a processor; and
a memory with computer code instructions stored thereon, the processor and the memory, with the computer code instructions, being configured to cause the system to:
a) apply a representation of the one or more test agents to a machine-learning classifier trained on a training dataset that comprises (i) a quantification of partitioning of training agents in an in vitro protein condensate that corresponds to the in vivo condensate and (ii) a representation of the training agents; and
b) quantify a partitioning of the one or more test agents in the in vivo condensate.

46. A non-transitory computer readable medium with instructions stored thereon for quantifying partitioning of one or more test agents in an in vivo condensate, the instructions, when executed by a processor, causing the processor to:
a) apply a representation of the one or more test agents to a machine-learning classifier trained on a training dataset that comprises (i) a quantification of partitioning of training agents in an in vitro protein condensate that corresponds to the in vivo condensate and (ii) a representation of the training agents; and
b) quantify a partitioning of the one or more test agents in the in vivo condensate.
